SEQUENCE LISTING (43 sequences)

SEQ ID NO: 1 - DNA, 20 bp, artificial sequence
  primer EEEV-1V(+)
atagggtatggtgtagaggc

SEQ ID NO: 2 - DNA, 44 bp, artificial sequence
  primer BeAr436087-T7-SacI(+)
ggagaggataatacgactcactatagatagggtatggtgtagag

SEQ ID NO: 3 - DNA, 21 bp, artificial sequence
  primer EEEV-366(+)
gacaaatgtattgcctctaag

SEQ ID NO: 4 - DNA, 20 bp, artificial sequence
  primer EEEV-900(+); where r at position 3 is a purine
gtraagaagattaccatcag

SEQ ID NO: 5 - DNA, 18 bp, artificial sequence
  primer EEEV-1948(-)
caatgtggtgtaagtaac

SEQ ID NO: 6 - DNA, 24 bp, artificial sequence
  primer EEEV-2050(+)
gatattgatgccagaaaatgcgtc

SEQ ID NO: 7 - DNA, 24 bp, artificial sequence
  primer EEEV-2480(+)
ccaaagaaagtggtattgtgtgga

SEQ ID NO: 8 - DNA, 24 bp, artificial sequence
  primer EEEV-3020(+)
gcagcgatgtttaccagaataaag

SEQ ID NO: 9 - DNA, 20 bp, artificial sequence
  primer EEEV-3720(-); where r at positions 7 and 13 is a purine
ctgatartggtgrtgcttgt

SEQ ID NO: 10 - DNA, 27 bp, artificial sequence
  primer EEEV-4440(-); where y at position 9 is a pyrimidine
gtttcccaytgtttgtccagacagtag

SEQ ID NO: 11 - DNA, 29 bp, artificial sequence
  primer EEEV-4740(-); where m at position 11 is an adenine or a cytosine and y at positions 12,15 and 18 is a pyrimidine
gagagtatgamyagyatycgctctaagtg

SEQ ID NO: 12 - DNA, 17 bp, artificial sequence
  primer EEEV-4908(+)
ctgtaatcctgtatttc

SEQ ID NO: 13 - DNA, 18 bp, artificial sequence
  primer EEEV-5050(-); where r at position 8 is a purine and y at position 11 is a pyrimidine
gacgtccrgcyccaccag

SEQ ID NO: 14 - DNA, 22 bp, artificial sequence
  primer EEEV-6510(-)
tccattacgaacctatccattg

SEQ ID NO: 15 - DNA, 23 bp, artificial sequence
  primer EEEV-6896(-), where K at position 6 is a guanine or thymine and S at position 9 is a guanine or cytosine
aagcgkgtscctgtaggtaagtg

SEQ ID NO: 16 - DNA, 18 bp, artificial sequence
  primer EEEV-7514(+)
taaccctctacggctgac

SEQ ID NO: 17 - DNA, 20 bp, artificial sequence
  primer EEEV-7514(+)
ttaggtcagccgtagagggt

SEQ ID NO: 18 - DNA, 21 bp, artificial sequence
  primer EEEV-8860(-)
cattgagccaggatgtaatag

SEQ ID NO: 19 - DNA, 23 bp, artificial sequence
  primer EEEV-8710(+); where m at position 12 is an adenine or cytosine and y at position 15 is a pyrimidine
agagatttggamacycatttcac

SEQ ID NO: 20 - DNA, 18 bp, artificial sequence
  primer EEEV-9210(-)
cacttcctgttgtcaatc

SEQ ID NO: 21 - DNA, 27 bp, artificial sequence
  primer EEEV-9010(-)
gatcaaggccattatgtagaaatgcac

SEQ ID NO: 22 - DNA, 18 bp, artificial sequence
  primer EEEV-9792(+)
cagacgacaccttgcaag

SEQ ID NO: 23 - DNA, 26 bp, artificial sequence
  primer EEEV-10850(-)
gtgcactcagtaattttacattccag

SEQ ID NO: 24 - DNA, 18 bp, artificial sequence
  primer EEEV-11599(-)
aaaagacagcattatgcg

SEQ ID NO: 25 - DNA, 59 bp, artificial sequence
  primer T25-NotI(-)
gcggccgctttttttttttttttttttttttttgaaatattaaaaacaaaataaaaaca

SEQ ID NO: 26 - DNA, 28 bp, artificial sequence
  primer EEE-SA-11,157 to construct pM1-EEEV-NA/SA
ccacaagcttaccagcgtagtcacctgc

SEQ ID NO: 27 - DNA, 35 bp, artificial sequence
  primer EEE-SA(E1)/NA(3′)-R to construct pM1-EEEV-NA/SA
tatgtggttgacaagatgttagtgtttgtgggtga

SEQ ID NO: 28 - DNA, 35 bp, artificial sequence
  primer EEE-SA(E1)/NA(3′)-F to construct pM1-EEEV-NA/SA
tcacccacaaacactaacatcttgtcaaccacata

SEQ ID NO: 29 - DNA, 29 bp, artificial sequence
  primer pGEM-R to construct pM1-EEEV-NA/SA
actcaagctatgcatccaacgcgttggga

SEQ ID NO: 30 - DNA, 27 bp, artificial sequence
  primer EEE-NA-11,068 to construct pM1-EEEV-SA/NA
ccacaagcttcactgcaaacatccatc

SEQ ID NO: 31 - DNA, 35 bp, artificial sequence
  primer EEE-NA(E1)/SA(3′)-R to construct pM1-EEEV-SA/NA
ggtaatttactgctagtattaatgtctatggaaga

SEQ ID NO: 32 - DNA, 35 bp, artificial sequence
  primer EEE-NA(E1)/SA(3′)-F to construct pM1-EEEV-SA/NA
tcttccatagacattaatactagcagtaaattacc

SEQ ID NO: 33 - DNA, 29 bp, artificial sequence
  primer pGEM-R to construct pM1-EEEV-SA/NA
actcaagctatgcatccaacgcgttggga

SEQ ID NO: 34 - RNA, 28 nt, BeAr 436087 strain
  hair pin loop E of BeAr 436087 strain
gggcuaaucgcuacagcagugguugccc

SEQ ID NO: 35 - RNA, 72 nt, BeAr 436087 strain
  hair pin loops C1 and C2 of BeAr 436087 strain
cuauauuauaucaacauaguucgauguacuuccgagcuauguugcugguggugcauaaugccaccgguguag

SEQ ID NO: 36 - RNA, 74 nt, BeAr 436087 strain
  hair pin loops B1, B2 and B2′ of BeAr 436087 strain
caucguaguucgauguacuuccgagcuacgaugaaggugguguauaacgccaccaaagcagugcauaaugcugc

SEQ ID NO: 37 - RNA, 19 nt, BeAr 436087 strain
  hair pin loop A2 of BeAr 436087 strain
ggcagcgcauaaugcugcc

SEQ ID NO: 38 - RNA, 35 nt, BeAr 436087 strain
  sequence of BeAr 436087 strain that does not have hairpin loop
cuuuuauguuuuuauuuuguuuuuaauauauuuca

SEQ ID NO: 39 - RNA, 69 nt, FL93-939 strain
  hair pin loops F and E of FL93-939 strain
ggaggaacaucagcauuccuuguucuggggcuuauugcuacagcaguggugcccuaguuuuguucuucc

SEQ ID NO: 40 - RNA, 70 nt, FL93-939 strain
  hair pin loops C1 and C2 of FL93-939 strain
cacuaaauucaccuaguucgauguacuuccgagcuaugugacgguggugcauaaugccgccgaugcagug

SEQ ID NO: 41 - RNA, 21 nt, FL93-939 strain
  hair pin loop B2 of FL93-939 strain
gggcagugcauaaugcugcuc

SEQ ID NO: 42 - RNA, 21 nt, FL93-939 strain
  hair pin loop A2 of FL93-939 strain
aggcagcgcauaaugcugucu

SEQ ID NO: 43 - RNA, 35 nt, FL93-939 strain; identical to SEQ ID NO: 38
  3′ UTR of FL93-939 strain without the loop

(19) United States
(12) Patent Application Publication
(10) Pub. No.: US 2010/0247565 A1
(43) Pub. Date: Sep. 30, 2010

(54) Chimeric sindbis-eastern equine encephalitis virus and uses thereof

(76) Inventors: Ilya Frolov, Galveston, TX (US); Patricia V. Aguilar, New York, NY (US); Scott C. Weaver, Galveston, TX (US); Slobodan Paessler, Galveston, TX (US)

Correspondence Address: Benjamin Aaron Adler;ADLER & ASSOCIATES, 8011 Candle Lane, Houston, TX, 77071, US

(21) Appl. No.: 11/478,068

(22) Filed: Jun. 29, 2006

Related U.S. Application Data
(60) Provisional application No. 60/695,735, filed on Jun. 29, 2005.

(51) Int. Cl. A61K 39/193; A61P 31/14; C12N 7/00; C12N 15/74; C12N 5/10; C12Q 1/70

(52) U.S. Cl. 424/199.1; 435/235.1; 435/320.1; 435/325; 435/5

(57) ABSTRACT

The present invention discloses a chimeric alphavirus comprising a Sindbis virus cDNA fragment and an Eastern equine encephalitis virus cDNA fragment. The present also discloses the use of this chimeric alphavirus as vaccines and in serological and diagnostic assays.

### CROSS REFERENCE TO RELATED APPLICATION

This non-provisional application claims benefit of provisional application U.S. Ser. No. 60/695,735 filed on Jun. 29, 2005, now abandoned.

### FEDERAL FUNDING LEGEND

This invention was produced in part using funds obtained through a award U54 AI057156 from the National Institute of Allergy and Infectious Disease. Consequently, the federal government has certain rights in this invention.

### BACKGROUND OF THE INVENTION

1. Field of the Invention

The present invention relates to the fields of molecular biology, virology and immunology. More specifically, the present invention provides an attenuated recombinant chimeric sindbis-eastern equine encephalitis virus (EEEV) and discloses its use as vaccines and in serological and diagnostic assays.

2. Description of the Related Art

Eastern equine encephalitis virus (EEEV) was first identified as a distinct etiologic agent of central nervous system (CNS) disease following the isolation from brain tissue of horses in 1933 (Glitner 1933; TenBroeck 1933) and a human in 1938 (Fothergill 1938a). It is a positive sense RNA virus that possesses a genome of approximately 11.7 kb, capped at the 5′ end and polyadenylated at the 3′ end. The genome encodes four nonstructural proteins (nsp1-4) that are important for virus replication and polyprotein processing and three structural proteins (capsid and the envelope proteins E1 and E2) that are involved in receptor recognition, virus attachment, penetration of virus into the cells and fusion of viral and cellular membranes.

Since its first isolation, sporadic epizootics in horses and outbreaks in humans have been reported in the eastern United States, Central and South America. In North America, the EEEV enzootic transmission cycle involves wild birds and the ornithophilic mosquito vector Culiseta melanura (Grimstad 1983). In South America, the transmission cycle has not been described as much in detail as in North America. However, based on virus isolations and experimental transmission experiments Culex (Melanoconion) spp. mosquitoes are suspected enzootic vectors (Shope 1966). The enzootic cycle in South America may also involve rodents and birds. Although very little is known about the vectors involved in the epizootic cycle in South America, Culex nigripalpus may function as bridge vectors between the enzootic and epizootic cycles (Scott 1989).

Thus, EEEV is currently considered the most deadly among the mosquito-borne viruses due to high mortality rate associated with apparent infection, which is up to 90% in horses. In humans, it has been estimated that the fatality rate following symptomatic infection approaches 80% and many survivors have crippling sequellae (mental retardation, convulsions, paralysis). Although the number of human cases is relatively low, EEEV has a strong social and economic impact in USA due to the high cost associated with mosquito control, prevention and surveillance. More recently, an increase in the number of equine EEEV cases in the past 2 years has raised concern in the general population and demonstrates its continuing importance as an emerging arboviral threat. Additionally, EEEV is a category B priority agent of the National Institute of Allergy and Infectious Disease due to its virulence, potential use as a biological weapon, and the lack of a licensed vaccine or effective treatments for human infection.

Previous studies using serological methods have recognized two antigenic EEEV varieties: North (NA) and South American (SA) (Calisher 1988; Calisher 1980; Casals 1964). These varieties exhibit important biological differences in their transmission cycles and virulence. In general, EEEV strains from central and South America appear to be less virulent than North American strains. The former occasionally cause disease and death in horses but human infections are rarely detected and seldom result in overt neurological disease; human infections with the NA strains are believed to often result in disease with neurological complications (Scott 1989; Walder 1980). Infection of humans in the Amazon basin was demonstrated during seroprevalence studies (Causey et al., 1958; Alice, 1956). Although EEEV was implicated in fatal equine epizootics in Braganca, Para State, no neurological disease in humans was reported during these outbreaks (Causey 1962; Travassos da Rosa 1998; Vasconcelos 1998). EEEV was also isolated repeatedly in Argentina from sick or dead horses between 1930 and 1958 and the virus was presumably responsible for at least 3 outbreaks in 1976, 1981 and 1988, based on serological diagnoses. However, no human neurological disease was reported during epizootic periods despite active surveillance and seroprevalence levels of up to 66% in some locations (Sabattini 1998). The reason for this apparent difference in human virulence is still unknown.

In experimentally infected laboratory mice, EEEV produces a neurological disease that resembles human and equine infections. Virus is detected in the brain as early as day 1 PI in some cases (Vogel et al., 2005) and signs of murine disease include ruffled hair, anorexia, vomiting, lethargy, posterior limb paralysis, convulsions and coma. Histopathological studies have revealed extensive involvement of the brain with neuronal degeneration, cellular infiltration and perivascular cuffing, which are also common pathological changes observed in the human central nervous system (CNS). Thus, understanding potential mechanism of EEEV virulence in the mouse model could aid in the understanding of EEEV human virulence.

Furthermore, very little is known about the genetic determinants that are crucial for EEEV neurovirulance and extremely important and necessary especially for effective vaccine development. For instance, studies with other alphaviruses have suggested both structural and nonstructural genes to be important for alphavirus virulence. Most of these studies have focussed on the structural proteins particularly the E2 glycoprotein gene. The contributions of several individual mutations in E1 and E2 glycoproteins to Venezuelan Equine Encephalitis Virus (VEEV) virulence in mice have been well characterized (Bernard 2000; Davis et al., 1991; Grieder et al., 1995). A single mutation in the E2 glycoprotein of the Venezuelan Equine Encephalitis Virus Trinidad Donkey strain (TRD) conferred a delay in replication of the mutant virus in mice and reduced the virulence of the virus (Davis et al., 1991). Additionally, two viral determinants, glycoproteins and the 5′UTR were shown to be responsible for the IFN resistant phenotype of the Trinidad Donkey strain (Spotts et al., 1998). Later, the importance of the 5′UTR in Venezuelan Equine Encephalitis Virus was demonstrated when a virus with a single mutation in this region resulted in an avirulence in mice and reduced growth in cell culture (White et al., 2001). More importantly, studies with chimeric viruses demonstrated that the E2 glycoprotein was the site of the epitopes that defined the enzootic and epizootic subtypes as well as mosquito infectivity in Venezuelan Equine Encephalitis Virus (Brault et al., 2002; Brault et al., 2004; Weaver et al., 2004). In addition, both structural and nonstructural genes have also been implicated as contributing factors for the epizootic phenotype and for guinea pig virulence.

Several studies with Sindbis virus demonstrated that amino acid changes in the envelope glycoproteins were associated with changes in neurovirulance (Lustig et al., 1988; Dropulic et al., 1997; Polo & Johnston 1990; Tucker & Griffin 1991; Tucker et al., 1993). Single mutations in the E1 and E2 glycoproteins resulted in mutants with an attenuated infection phenotype in neonatal mice (Davis et al., 1996; Polo & Johnston 1990). Recently, studies with Semliki forest virus and Sindbis virus demonstrated the importance of nsP1, nsP2 and nsP3 in alphavirus virulence. Inhibition of palmitoylation of nsP1 attenuated SFV neurovirulance (Ahola et al., 2000), whereas mutation of the opal codon to arginine in nsP3 increased the virulence of a previously identified avirulent strain of SFV. When several amino acids in the nsP3 region were introduced th attenuated strain was fully restored to neurovirulance providing strong evidence for the role of nsP3 in SFV pathogenesis (Tuittila & Hinkkanen 2003). Similarly, mutations in the nsP1 and nsP2 dramatically increased virulence in SFV, further supporting the role of the nonstructural proteins in pathogenesis (Tuittila et al., 2000). Moreover, in Sindbis virus, nsP2 was found to have a role in suppressing the IFN response in infected cells (Frolova et al., 2002).

Thus, prior art is deficient is deficient in understanding EEEV pathogenesis, role of the structural and non structural genes in EEEV virulence and use of these genes in the development of vaccines and antiviral drugs. The present invention fulfills this long-standing need and desire in the art.

### SUMMARY OF THE INVENTION

In one embodiment of the present invention, there is a DNA encoding a chimeric Eastern equine encephalitis virus (EEEV) comprising a Sindbis virus cDNA fragment and the Eastern equine encephalitis virus cDNA fragment. A described herein, a host cell comprising and expressing the vector and an attenuated EEEV comprising the DNA described herein.

In yet another related embodiment of the present invention, there is a pharmaceutical composition comprising the above-mentioned attenuated Eastern equine encephalitis virus and a pharmaceutically acceptable carrier. In a related embodiment of the present invention, there is an immunogenic composition comprising a live attenuated EEEV vaccine, where the vaccine comprises the attenuated Eastern equine encephalitis virus described herein. In a further related embodiment of the present invention, there is an immunogenic composition comprising an inactivated vaccine, where the vaccine comprises the attenuated Eastern equine encephalitis virus described herein that is inactivated.

In another embodiment of the present invention, there is a method of protecting an individual from infections resulting from exposure to Eastern equine encephalitis virus. Such a method comprises administering a pharmacologically effective amount of the immunogenic composition comprising the live attenuated Eastern equine encephalitis virus vaccine described herein, where the vaccine elicits an immune response against the Eastern equine encephalitis virus in the individual, thereby protecting the individual from the infection.

In yet another embodiment of the present invention, there is a method of protecting an individual from infections resulting from exposure to Eastern equine encephalitis virus. Such a method comprises administering a pharmacologically effective amount of the immunogenic composition comprising the inactivated EEEV vaccine described herein, where the vaccine elicits an immune response against the EEEV in the individual thereby protecting the individual from the infection.

In still yet another embodiment of the present invention, there is a method of determining the presence of an antibody to EEEV in a subject. Such a method comprises obtaining serum sample from the subject and performing assay using the attenuated EEEV described herein to determine presence or absence of antigenic reactions, effect of physical properties of the EEEV or a combination thereof in the serum sample, thereby determining the presence of the antibody to EEEV in the subject. In another embodiment of the present invention, there is a method of determining the presence of an antibody to EEEV in a subject. Such a method comprises obtaining serum sample from the subject and performing assay using an inactivated EEEV, where the inactivated EEEV comprises the attenuated EEEV described herein that is inactivated to determine presence or absence of antigenic reactions, effect of physical properties of the EEEV or a combination thereof in the serum sample, thereby determining the presence of the antibody to EEEV in the subject. In yet another embodiment of the present invention, there is a kit. Such a kit comprises the attenuated Eastern equine encephalitis virus described herein, the attenuated Eastern equine encephalitis virus described herein that is inactivated or combinations thereof.

### BRIEF DESCRIPTION OF THE DRAWINGS

FIG. 1 shows survival curve in 5-7 week old mice infected subcutaneously with virulent and avirulent EEEV strains (N=10).

FIG. 2 shows survival curve in 5-7 week old NIH Swiss mice infected intracranially with 10⁶ PFU of virulent and avirulent EEEV strains (N=5).

FIG. 3 shows viremia levels in mice infected with EEEV strains. Strain BeAr 436087, which does not cause mortality in mice replicated to about 10-fold higher levels than the other EEEV strains.

FIG. 4 shows titers of virus in different organs obtained from NIH Swiss mice infected subcutaneously with 1000 PFU of avirulent strain (BeAr436087). The Y axis indicates assay sensitivity limits. Bars indicate the standard error.

FIG. 5 shows titers of virus in different organs obtained from NIH Swiss mice infected subcutaneously with 1000 PFU of virulent strain (792138). The Y axis indicates assay sensitivity limits. Bars indicate the standard error.

FIGS. 6A-6B show viremia levels in 129 Sv/Ev wild-type versus IFN-α/β (FIG. 6A) and IFN-γ receptor deficient knock out mice (FIG. 6B). Bars indicate the standard error.

FIGS. 7A-7B show survival in 129Sv/Ev wild type versus IFN-α/β (FIG. 7A) and IFN-γ receptor deficient knock out mice (FIG. 7B).

FIGS. 8A-8B show predicted secondary structure based on the 3′ end UTR of the avirulent BeAr 436087 (FIG. 8A) and virulent strain FL93-939 (FIG. 8B). Two extra hairpin loop structures (B1 and B2) are observed in the avirulent strain. The nucleotide sequence representing the structure of the BEAr strain are identified as follows: Loop E (SEQ ID NO: 34), Loops C1 and C2 (SEQ ID NO: 35), Loops B1, B2 and B2′ (SEQ ID NO: 36), Loop A2 (SEQ ID NO: 37) and the sequence without the loop (SEQ ID NO: 38). The nucleotide sequence representing the structure of the FL93-939 strain are identified as follows: Loops E and F (SEQ ID NO: 39), Loops C1 and C2 (SEQ ID NO: 40), Loop B2 (SEQ ID NO: 41), Loop A2 (SEQ ID NO: 42) and the sequence without the loop (SEQ ID NO: 43).

FIG. 9 is a schematic representation of the strategy used to amplify and sequence the complete genome of the NA strain FL-93-939. Vertical lines indicate the restriction sites used to incorporate the fragments into the final construct to create the NA infectious clone.

FIGS. 10A-10B show genetic organization of the NA/SA (FIG. 10A) and SA/NA (FIG. 10B) chimera constructs.

FIGS. 11A-11B show the virus replication curve of FL93-939 parental and infectious clone virus in Vero cells (FIG. 11A) and in C710 mosquito cells (FIG. 11B). Error bars indicate the standard error.

FIG. 12 shows replication of parental and infectious clone virus in 5-6 week old NIH Swiss mice. No significant difference in replication was observed between the viruses (P<0.05). Error bars indicate the standard error.

FIG. 13 shows survival curve in 5-6 week old NIH Swiss mice infected with FL93-939 parental (n=5) and infectious clone (I.C.) virus (n=6).

FIGS. 14A-14B show replication of NA/SA, SA/NA chimera and parental viruses in Vero cells (FIG. 14A) and in C710 mosquito cells (FIG. 14B). Error bars indicate the standard error.

FIG. 15 shows survival curve of 5-7 week old mice infected subcutaneously with 1000 PFU of NA/SA, SA/NA and parental EEEV.

FIG. 16 shows daily viremia levels in mice infected with NA/SA, SA/NA and parental viruses. Bars represent standard errors.

FIG. 17 is a diagrammatic representation of a DNA encoding a chimeric alphavirus. This DNA fragment comprises of Sindbis virus cDNA fragment and EEEV cDNA fragment. The Sindbis virus cDNA fragment comprises of cis-acting sequences from 5′ and 3′ termini, 26S promoter and nonstructural protein genes (nsP1, nsP2, nsP3 and nsP4). The EEEV cDNA fragment comprises structural protein genes (E1, E2). Representative EEEV strains used for the structural proteins are FL93-939 or BeAr436087.

FIG. 18 shows a schematic representation of the different chimeras used herein.

FIGS. 19A-19C show results of Sindbis-EEE chimeric virus studies in murine models. FIG. 19A shows attenuation of the Sindbis-EEE chimeric viruses in 6 day old mice. FIG. 19B shows efficacy of SIN/EEE-North American vaccine candidate. FIG. 19C shows efficacy of SIN/EEE-South American vaccine candidate.

FIGS. 20A-20B show results of Sindbis-EEE chimeric virus studies in equine model. FIG. 20A shows effect on equine viremia after challenge. FIG. 20B shows equine febrile responses to challenge.

### DETAILED DESCRIPTION OF THE INVENTION

Although epidemiological and clinical studies revealed information regarding the transmission cycles and pathogenesis of EEEV in humans, equines and other animals, the viral genetic determinants that confer the neurovirulant phenotype to EEEV were not known. Previous studies focused on the pathogenesis of attenuated variants of EEEV that were produced after extensive animal or cell culture passages from small plaques mutants that arose either spontaneously or after induction by chemical mutagens (Brown 1975; Dremov 1978; Solyanik 1972). However, these studies were based on artificially created virus mutants and therefore the results do not reflect infection with a natural strain of the virus. The currently used equine vaccines are formalin inactivated preparations made from virulent, wild type North American (NA) strains. These vaccines are used to vaccinate other domesticated animals (pigs, emus, pheasants etc) and occasionally wild animals that develop the disease. Moreover, nothing has been described about potential genes that may be involved in a natural attenuation phenotype of EEEV due to the lack of a naturally attenuated strain of EEEV that was unable to cause fatal disease in animal models. All of this has resulted in lack of a licensed human EEEV vaccine.

The present invention described the phenotypic and genetic characterization of a strain of EEEV, isolated from mosquitoes that was unable to cause fatal disease in the mouse model. It also demonstrated that the attenuated strain replicated in the brain but was cleared from all organs including the brain by day 6 post infection. Additionally, this strain caused mild focal encephalitis without signs of clinical infection in the animals even after intracranial inoculation. In distinct contrast, replication of all other EEEV strains in the brain increased over time and achieved the highest titer at the time of death due to encephalitis.

Additionally, immunohistochemical analyses confirmed the replication of the attenuated strain in certain neuron populations. However, they were located in a small focus of the brain, suggesting that the attenuated strain replicated in some neurons, but was unable to efficiently spread to adjacent neurons or cause disseminated encephalitis. In distinct contrast, the virulent strain, which also replicated in neurons, initiated the infection in small perivascular foci, probably the site of virus invasion into the CNS and rapidly disseminated within the brain resulting in acute, disseminated encephalitis and the death of the animals.

Studies with other viruses had shown that avirulence in adult mice correlated with restriction of viral replication by central nervous system cells (Sharpe et al., 1990; Swoveland et al., 1989). This was also observed in mice infected with an attenuated strain of the alphavirus SFV, which had restricted replication in neurons and oligodendrocytes (Fazakerley et al., 1993). Electron microscopy studies showed that complete virus particles were not observed in the neurons or oligodendrocytes of adult mice infected with the avirulent SFV strain due to restriction in virus assembly. Whether this could explain the limitation in virus spread within the brain in animals infected with the attenuated strain of EEEV is not yet known.

The attenuated strain identified in the present invention induced the highest viremia levels in mice compared to other EEEV strains. Previous studies with VEEV had demonstrated that higher viremia correlated with neurovirulance, since enzootic ID viruses, which were unable to cause a high mortality in horses, developed low viremia levels in equines (2.4 log₁₀ SmicLD₅₀/ml), whereas epizootic strains of VEEV, which caused a high mortality in horses, usually developed higher (5.3-7 log₁₀ SmicLD₅₀/ml) and longer viremia than enzootic viruses (Wang et al., 2001; Weaver et al., 2004). However, the present invention demonstrated that the viremia levels in mice infected with EEEV did not correlate with neurovirulance. The attenuated strain induced more than 10-fold higher murine viremia, yet did not cause apparent central nervous disease (CNS) as opposed to other EEEV strains. Whether higher viremia in mice infected with EEEV induced a more potent immune response in the animals will be further examined.

The present invention also demonstrated no difference in the appearance and levels of neutralizing antibodies. Moreover, mice deficient in Type I and II IFN response were also resistant to infection with the attenuated strain like wild type mice, thereby suggesting that the attenuation of the avirulent strain was not dependent on Type I or Type II IFN. Further studies will be performed in order to determine the potential role of T cells in the clearance of the avirulant strain from neuron populations and/or whether the attenuated strain caused persistent infection in the brain. The present invention demonstrated that mice infected with the avirulent strain were observed for up to three months post-infection did not develop any neurological signs. Moreover, the animals were completely protected against the fatal disease when they were challenged one to three months post-infection with more virulent strains of EEEV, thereby suggesting that the avirulent strain produced a long lasting immunity against EEEV.

Furthermore, although previous studies provided insights regarding the role of structural and nonstructural genes in the virulence of alphavirus, the role of these genes in EEEV pathogenesis is not known. Moreover, although VEEV and EEEV cause encephalitis in the laboratory murine model, these viruses differ in the pathogenesis. For instance, EEEV is mainly a neurotropic virus whereas VEEV is neurotropic but also causes biphasic pathogenesis with systemic infection and pathological changes in the lung and lymphoid tissue of the gastrointestinal tract, spleen and peripheral nodes. Additionally, the mechanism by which these viruses enter the central nervous system might also be different. For example, VEEV invades the brain of the mice via the olfactory bulb (Charles et al., 1995), whereas EEEV is contemplated to cross the blood brain barrier by passive transfer or within infected leukocytes and that the olfactory bulb is not an important route of neuroinvasion for EEEV (Vogel et al., 2005). Similarly, EEEV causes a different disease in the mouse model than SFV and Sindbis virus and therefore extrapolation of the genetic studies with these other alphaviruses may not necessarily correlate with the genetic determinants of EEEV virulence.

The present invention used a newly created infectious clone of a highly virulent NA strain of EEEV as a backbone to construct two chimeric viruses harboring the structural and nonstructural genes of recently identified avirulent EEEV strains. The results demonstrated that both chimeras were able to induce neurological disease in the animals and to cause mortality, thereby suggesting that both structural and nonstructural genes of EEEV were important contributors for neurovirulance. However, the possibility that the 5′ and 3′ UTR contributed to the neurovirulance phenotype of the chimeras cannot not be excluded.

It is known that the nonstructural proteins form essential components of alphaviruses RNA replication and transcription complexes (Strauss & Strauss 1994a). The results obtained in the present invention with the chimeras support these previous observations. The chimera harboring the nonstructural genes of the SA avirulent strain induced similar viremia levels as the virulent strain, thus both viruses produced more than 10-fold higher viremia in mice than the reciprocal chimera and the NA virulent strain. Similarly, the chimera harboring the nonstructural genes of the NA strains produced comparable viremia titers as the NA strain. The present invention also demonstrated that the viremia levels did not correlate with EEEV neurovirulance. Thus, it is necessary to investigate more highly defined viral genetic determinants to understand the mechanism of EEEV neurovirulance, which will be helpful to develop live-attenuated EEEV vaccine.

Furthermore using a completely different strategy for attenuation, the present invention developed infectious cDNA clones encoding chimeric alphaviruses that could be used as live attenuated vaccine strains and as diagnostic reagents. These chimeric alphavirus strains included the cis-acting sequences from the 5′ to 3′ termini, the 26S promoter and the nonstructural protein genes of the Sindbis virus genome. The structural protein genes were derived from 2 strains (FL93-939 and BeAr436087) of eastern equine encephalitis viruses (EEEV). The virus particles produced from such chimeric strains had protein content that was identical to the wild-type EEEV.

The present invention also demonstrated that these chimeric virus strains replicated to high titer in cell cultures but produced no detectable disease when injected intracerebrally at high doses into mice. Instead the chimeric strains induce the production of neutralizing antibodies and protected the mice from lethal challenge with EEEV. Additionally, these chimeric strains also served as surrogates for wild type EEEV in several serological assays.

Thus, although the protein content was identical to wild-type EEEV, these strains were highly attenuated to offer vaccine and reagent safety. Furthermore, although they elicited immune responses like the wild type EEEV strains and reacted identically in antibody assays, they were not considered select agents and could be manipulated at biosafety level 2. Thus, the alphaviruses of the present invention differed significantly from the previously known chimeric alphaviruses. Additionally, the present invention also demonstrated that vaccination of horses, mice and hamsters with Sindbis-EEE chimeric viruses induced production of antibody in the vaccinated animals. The efficacy of these chimeric viruses mined by performing immunization and challenge experiments in these animals.

Based on the properties of these chimeric as described supra, it is contemplated that these chimeric viruses can be used as live-attenuated vaccines in humans or domestic animals. Additionally, these viruses can also be used in any experiments or assays that measure antigenic reactions or other physical properties of EEEV virus particles due to the similarity in the protein content of the chimeric viruses and the wild type EEEV. Such assays include but are not limited to serological assays such as plaque reduction neutralization tests, enzyme linked immunosorbent assays, hemagglutination inhibition and complement fixation assays conducted with live or inactivated antigens produced from the chimeras, production of virus for inactivation using formalin for vaccination of humans or animals and structural studies employing methods such as electron microscopy.

The more important and immediate use of these virus strains would be in the production of formalin-inactivated EEEV vaccines, which currently requires vaccination of employees that is expensive and requires frequent boosters, select agent security measures and expensive biological containment. Additionally, the inactivation of wild type, virulent EEEV is technically challenging and the presence of live virus in a vaccine lot can result in encephalitis in the vaccinated animal. The chimeras of the present invention are safer and cheaper to produce and safe even if the inactivation is incomplete. Furthermore, an equine vaccine company could substitute these chimeric viruses into their production protocol without any methodological changes. Although the present invention has generated chimeric virus strains that comprise EEEV, same principle as discussed herein may be applied to construct chimeric virus strains that comprise other alphaviruses (Venezuelan equine encephalitis virus (VEEV) or Western equine encephalitis virus (WEEV)) or other related viruses. If modified accordingly, these chimeric viruses may then be utilized in the same way as is discussed for the chimeras of the present invention.

In summary, the present invention demonstrated that an attenuated strain of EEEV, BeAr436087 differing in virulence from all other strains tested in chimeric alpha viruses and isolated from a mosquito pool in Brazil caused no mortality in mice. Furthermore, the present invention also demonstrated that both the structural and non-structural genes of the virus were important for EEEV virulence in the mouse model by constructing an infectious cDNA clone of NA strain, which caused 80-90% mortality in mice along with two chimeric viruses that combined the structural and nonstructural genes of the virulent and avirulent strains. Additionally, chimeric alphaviruses of the present invention comprised of a combination of these clones and the Sindbis virus and had a protein content similar to the wild type EEEV. Although the protein content was similar, these chimeric viruses were highly attenuated and safe to use. Hence, it is contemplated that these strains could replace wild type Eastern equine encephalitis virus in current inactivated veterinary vaccine preparations to reduce cost and improve safety in production facilities as well as to improve safety against occasional presence of live virus in vaccine lots that can result in encephalitis. They also can be used in live form to allow single dose vaccination for faster and longer lasting immunity (probably life-long; in contrast to the current vaccine that requires multiple initial doses and semiannual boosting to maintain protective immunity in horses). Additionally, these viruses can be used in diagnostic assays.

The present invention discloses an equine encephalitis virus comprising a Sindbis virus cDNA fragment and the EEEV cDNA fragment. Specifically, the Sindbis virus cDNA fragment comprises cis-acting sequences from the 5′ and 3′ termini, 26S promoter and nonstructural protein genes while the EEEV cDNA fragment comprises structural protein genes. Representative examples of the strains of EEEV from where the cDNA fragment is derived from may include but is not limited to FL93-939 or BeAr436087 strain. Furthermore, the chimeric DNA may have protein content that is identical to wild-type EEEV.

invention is also directed to a vector comprising DNA described herein, a host cell comprising and expressing the vector and an attenuated EEEV comprising the DNA described herein. The present invention is further directed to a pharmaceutical composition comprising the attenuated EEEV described supra and a pharmaceutically acceptable carrier.

The present invention is further directed to an immunogenic composition comprising a live attenuated EEEV vaccine, where the vaccine comprises the attenuated EEEV described herein. Alternatively, the present invention is directed to an immunogenic composition comprising an inactivated EEEV vaccine, where the vaccine comprises the attenuated EEEV described herein, where the attenuated EEEV is inactivated. The present invention is also directed to a method of protecting an individual for infections resulting from exposure to Eastern equine encephalitis virus, comprising administering a pharmacologically effective amount of an immunogenic composition comprising the live attenuated EEEV vaccine described herein, where the vaccine elicits an immune response against the EEEV in the individual thereby protecting the individual from the infections. Additionally, the individual that may benefit from such a treatment is a human or a domestic animal.

Alternatively, the present invention is also directed to a method of protecting an individual for infections resulting from exposure to Eastern equine encephalitis virus, comprising administering a pharmacologically effective amount of the immunogenic composition comprising the inactivated EEEV vaccine described herein, where the vaccine elicits an immune response against the EEEV in the individual thereby protecting the individual from the infections. Additionally, the individual that may benefit from such a treatment is a human or a domestic animal. Generally, the infections may arise due to natural exposure of from a bioterror attack.

The present invention is further directed to a method of determining the presence of an antibody to Eastern equine encephalitis virus in a subject, comprising: obtaining a serum sample from the subject, and performing an assay using the attenuated virus described herein to determine the presence or absence of antigenic reactions, effect on physical properties of the EEEV or a combination thereof in the serum sample, thereby determining the presence of antibody to EEEV in the subject. Examples of such assays are not limited to but may include enzyme linked immunosorbent assays, hemagglutination inhibition assay, complement fixation assay or plaque reduction neutralization assay. Additionally, the serum may be obtained from a human or a domestic animal.

Alternatively, the present invention is further directed to a method of determining presence of an antibody to Eastern equine encephalitis virus in a subject, comprising: obtaining a serum sample from the subject, and performing assay using an inactivated EEEV, where the inactivated EEEV comprises the attenuated virus described herein that is inactivated to determine the presence or absence of antigenic reactions, effect on physical properties of the EEEV or a combination thereof in the serum sample, thereby determining the presence of antibody to EEEV in the subject. All other aspects regarding the type of assays and the subject is as discussed supra.

The present invention is still further directed to a kit comprising: an attenuated Eastern equine encephalitis virus described herein, an attenuated Eastern equine encephalitis virus described herein that is inactivated or combinations thereof. Furthermore, the kit may also comprise attenuated and/or inactivated forms of other related chimeric viruses (VEEV, WEEV or any related viruses) that are constructed based on the same principles as discussed herein.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof.

The composition described herein can be administered independently, either systemically or locally, by any method standard in the art, for example, subcutaneously, intravenously, parenterally, intraperitoneally, intradermally, intramuscularly, topically, or nasally. Dosage formulations of the composition described herein may comprise conventional non-toxic, physiologically or pharmaceutically acceptable carriers or vehicles suitable for the method of administration and are well known to an individual having ordinary skill in this art.

The composition described herein may be administered independently one or more times to achieve, maintain or improve upon a therapeutic effect. It is well within the skill of an artisan to determine dosage or whether a suitable dosage of the composition comprises a single administered dose or multiple administered doses. An appropriate dosage depends on the subject's health, the induction of immune response and/or prevention of infection caused by EEE virus, the route of administration and the formulation used.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### Viruses

The viruses used in the present invention (Table 1) were provided by the University of Texas Medical Branch World Reference Center for Emerging Viruses and Arboviruses. The strains were isolated in Vero cells from mosquitoes, and were chosen for these studies due to their low passage histories. Stocks were prepared in mice to avoid selection for attenuated alphavirus mutants that occur following passage in cells expressing glycosaminoglycans (Bernard et al. 2000; Byrnes & Griffin 1998; Heil et al. 2001; Klimstra et al. 1998). One- to 3-day-old mice were inoculated intracranially with each virus strain and a 10% suspension of homogenized brain tissue was prepared after morbidity or mortality was observed. The titers of the virus stocks were determined by plaque assay in Vero cells.

TABLE 1
EEEV isolates used for experimental infections

| VIRUS | PLACE OF | YEAR OF | SUB- | | PASSAGE |
|---|---|---|---|---|---|
| STRAIN | ISOLATION | ISOLATION | TYPE | SOURCE | HISTORY * |
| GML | Panama | 1984 | SA II | Mosquito | V-2/SMB-1 |
| 903836 | | | | | |
| BeAr | Brazil | 1975 | SA III | Mosquito | V-1/SMB-2 |
| 300851 | | | | | |
| BeAr | Brazil | 1985 | SA | Mosquito | V-1/SMB-1 |
| 436087 | | | | | |
| 792138 | USA | 1979 | NA | Mosquito | C6/36- |
| | | | | | 1/SMB-1 |

### Example 2

### Infection of Mice and Hamsters

Five to 6-week-old NIH Swiss mice from Harlan Laboratories (Indianapolis, Ind.) were maintained under specific pathogen-free conditions. The animals were allowed to acclimate to the laboratory conditions for one week and then placed into cohorts of 5 for subcutaneous infection with EEE strains 792138, FL93-939, GML903836, BeAr 300851 and BeAr436087, and intracranial infection with EEEV strains 7921338 and BeAr 436087. Mice were subcutaneously infected with 1000 PFU of virus and intracranially infected with either 1000 PFU or 10E6 PFU of the same strains to compare the replication in the brain. The animals were bled daily (day 1-7) and monitored for clinical signs including fever, lethargy, paralysis or death for up to a month after infection. For the long-term antibody protection experiments, survivors were kept for up to three months and challenged with the EEEV strain 79-2138. To determine whether the attenuated BeAr 436087 strain was also avirulent in Gold Syrian hamsters, 5-7 and 12 week-old Gold Syrian hamsters were infected subcutaneously with 1000 PFU of the attenuated strain and the animals were monitored daily for signs of disease and mortality.

### Example 3

### Virus Replication In Vivo and Histopathology

Four animals infected subcutaneously and three mice infected intracranially were sacrificed daily (days 1 through 7) for pathogenesis studies. Briefly, animals were anesthetized and the thoracic cavity of each mouse opened to collect blood by cardiac puncture. Then, each animal was perfused with phosphate buffer saline (PBS) to eliminate the blood-associated virus and brain, heart, lung, spleen, liver and kidney were harvested for viral titration and histopathological studies. Tissues were homogenized to make a 10% suspension in EMEM containing 20% fetal bovine sera, penicillin streptomycin and glutamine (10 μg/ml). The final suspension was clarified by centrifugation and stored at −70° C. for virus titration by plaque assay in Vero cells. Blood samples were plaque assayed and a plaque reduction neutralization test (PRNT) was used to measure the antibody response. Tissues samples for histopathological studies were fixed in 4% paraformaldehyde in PBS for two days and then paraffin embedded, sectioned and stained with hematoxilin and eosin. Negative controls were tissues collected from mice inoculated with EMEM and processed in parallel.

### Example 4

### Immunohistochemistry Analysis

Immunohistochemistry was performed as described (Paessler 2004). Briefly, sections were deparaffined and rehydrated with xylene and graded ethanol solutions. Then, slides were treated with 3% hydrogen peroxide containing 0.05% sodium azide in PBS for 10 min followed by microwave antigen retrieval at 100° C. for 10 min in Dako Target retrieval solution in an H2800 microwave processor (Energy Beam Sciences, Agawan, Mass.). Slides were then incubated for 15 min in 0.1% avidin and 0.01% biotin (Vector Laboratories, Burlingame, Calif.), and for 30 min in 0.05% casein (Sigma, Saint Louis, Mo.)/0.05% Tween 20/PBS to block nonspecific protein binding. Murine hyperimmune sera against EEEV (produced by immunizing animals against NA and SA strains) were applied at 1:300 dilution to sections for 60 min. To provide an antibody negative control, the murine IgG-Ready to Use Kit (InnoGenex, San Ramon, Calif.) was used at the same IgG concentration, on infected tissue; the negative control included the brain of uninfected mice. The Histomouse-SP kit (Zymed laboratories, San Francisco, Calif.) was used for detection of mouse antibody. Slides were counterstained with Mayer's modified hematoxylin before mounting and microscopy studies.

### Example 5

### Infection of IFN α/β and γ Receptor Deficient Mice

Ten- to 13-week-old strain 129 Sv/Ev (wild type) mice were purchased from Jackson laboratories (Bar harbor, ME), and breeding pairs of the 129 Sv/Ev IFN-α/-β receptor −/− mice were generously provided by Herbert Virgin (Washington University, St Louis, Mo.) and allowed to breed under pathogen free conditions. Ten- to 13-week-old 129 Sv/Ev IFN-γ receptor −/− mice were purchased from Jackson laboratories and were allowed to acclimate to the laboratory conditions for one week. Mice were subcutaneously inoculated with 1000 PFU of EEEV strains 792138 and BeAr 436087 and bled 8, 24, 32, 48, 56, 72 and 96 hrs post-infection for viremia determination. The animals were observed daily for up to a month for clinical signs of illness and mortality.

### Example 6

### RNA Extraction, PCRs and Sequence Analysis Comparison

RNA was extracted from the virus stocks as described previously (Weaver 1999). A 250 μl volume of the 10% homogenized brain tissue was mixed with 750 μl of Trizol LS (Gibco-BRL, Gaithersburg, Md.) and RNA was extracted following the manufacturer's protocol. Reverse transcription was carried out in a 20 μl reaction containing 1 μM of antisense primer T25-NotI (−) or E/V 7514 (−), 1× First Strand Buffer (Gibco BRL, Gaithersburg, Md.), 1 mM dNTPs, 80 U RNAsin (Promega, Madison, Wis.), and 200 U of Superscript II reverse transcriptase (Gibco BRL, Gaithersburg, Md.). The cDNA was synthesized by incubating at 42° C. for 1 hr. The primers used for the PCRs are shown in Table 2. Briefly, PCRs were carried out by using 2.5 U of the high fidelity Pfu Turbo Polymerase (Stratagene, La Jolla, Calif.) in a 50 μl reaction containing 1×Pfu buffer, 300 nM of sense and antisense primer, 1 mM MgCl₂, 0.2 mM dNTPs, and 5 μl of the cDNA reaction. PCR amplification was carried out using 30 amplification cycles.

PCR amplicons were gel purified using the QIAquik Gel extraction kit (QIAGEN, Valencia, Calif.) and sequenced directly using the Big Dye terminator cycle sequencing ready reaction kit (Applied Biosystems, Foster City, Calif.) and 3.2 pmoles of primers. Sequences were aligned using the Mac Vector program (Accelrys Corporate, San Diego, Calif.).

TABLE 2
Primers used for amplification and sequencing
of the complete genome of EEEV strains.

| Primer | Sequence (5′→3′) (SEQ ID NO.) |
|---|---|
| EEEV-1V (+) | ATAGGGTATGGTGTAGAGGC |
| | (SEQ ID NO: 1) |
| BeAr436087- | GGAGAGGATAATACGACTCACTATAGATAGGGTA |
| T7-SacI (+) | TGGTGTAGAG |
| | (SEQ ID NO: 2) |
| EEEV-366 (+) | GACAAATGTATTGCCTCTAAG |
| | (SEQ ID NO: 3) |
| EEEV-900 (+) | GTRAAGAAGATTACCATCAG |
| | (SEQ ID NO: 4) |
| EEEV-1948 (−) | CAATGTGGTGTAAGTAAC |
| | (SEQ ID NO: 5) |
| EEEV-2050 (+) | GATATTGATGCCAGAAAATGCGTC |
| | (SEQ ID NO: 6) |
| EEEV-2480 (+) | CCAAAGAAAGTGGTATTGTGTGGA |
| | (SEQ ID NO: 7) |
| EEEV-3020 (+) | GCAGCGATGTTTACCAGAATAAAG |
| | (SEQ ID NO: 8) |
| EEEV-3720 (−) | CTGATARTGGTGRTGCTTGT |
| | (SEQ ID NO: 9) |
| EEEV-4440 (−) | GTTTCCCAYTGTTTGTCCAGACAGTAG |
| | (SEQ ID NO: 10) |
| EEEV-4740 (−) | GAGAGTATGAMYAGYATYCGCTCTAAGTG |
| | (SEQ ID NO: 11) |
| EEEV-4908 (+) | CTGTAATCCTGTATTTC |
| | (SEQ ID NO: 12) |
| EEEV-5050 (−) | GACGTCCRGCYCCACCAG |
| | (SEQ ID NO: 13) |
| EEEV-6510 (−) | TCCATTACGAACCTATCCATTG |
| | (SEQ ID NO: 14) |
| EEEV-6896 (−) | AAGCGKGTSCCTGTAGGTAAGTG |
| | (SEQ ID NO: 15) |
| EEEV-7514 (+) | TAACCCTCTACGGCTGAC |
| | (SEQ ID NO: 16) |
| EEEV-7514 (+) | TTAGGTCAGCCGTAGAGGGT |
| | (SEQ ID NO: 17) |
| EEEV-8860 (−) | CATTGAGCCAGGATGTAATAG |
| | (SEQ ID NO: 18) |
| EEEV-8710 (+) | AGAGATTTGGAMACYCATTTCAC |
| | (SEQ ID NO: 19) |
| EEEV-9210 (−) | CACTTCCTGTTGTCAATC |
| | (SEQ ID NO: 20) |
| EEEV-9010 (+) | GATCAAGGCCATTATGTAGAAATGCAC |
| | (SEQ ID NO: 21) |
| EEEV-9792 (+) | CAGACGACACCTTGCAAG |
| | (SEQ ID NO: 22) |
| EEEV 10850 (−) | GTGCACTCAGTAATTTTACATTCCAG |
| | (SEQ ID NO: 23) |
| EEEV 11599 (−) | AAAAGACAGCATTATGCG |
| | (SEQ ID NO: 24) |
| T25-Not I (−) | GCGGCCGCTTTTTTTTTTTTTTTTTTTTTTTTTGAA |
| | ATATTAAAAACAAAATAAAAACA |
| | (SEQ ID NO: 25) |

### Example 7

### RNA Folding

The secondary structure of the 5′ and 3′ end was predicted using the mfold program (Zuker 2003).

### Example 8

### Statistical Analysis

Statistical comparisons were performed using the paired Student's T test (Graph Pad, La Jolla, Calif.) to determine if differences in tissue titers between the strains were significant. Values of p≦0.05 were considered significant.

### Example 9

### Results

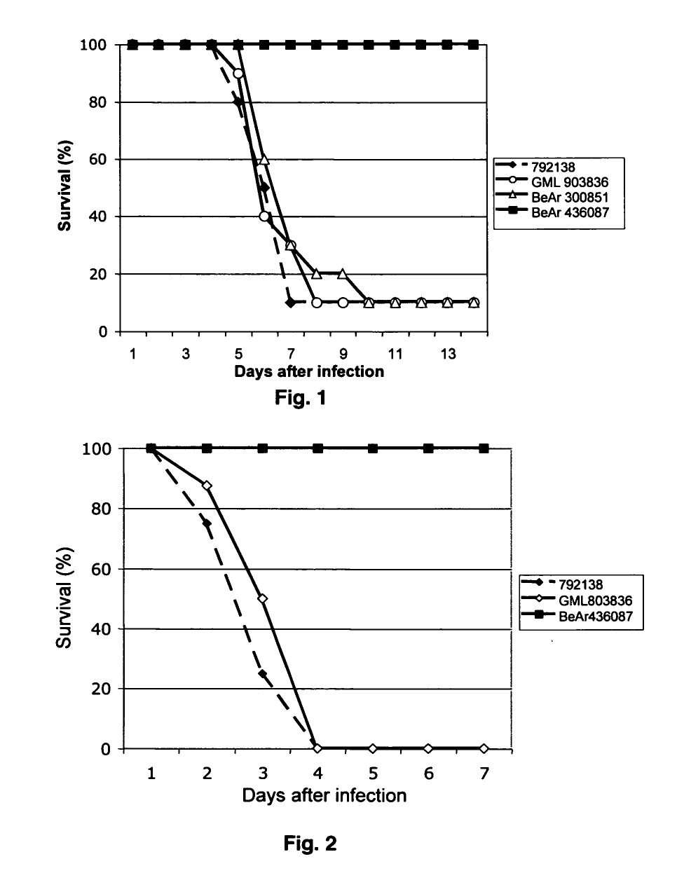

It was observed that of the 5-7 week old NIH Swiss mice infected subcutaneously with 1000 PFU of EEEV strains, mice infected with the BeAr 436087 strain (avirulent strain) survived the infection and did not develop any apparent sign of illness as opposed to mice infected with other EEEV strains (mean survival time (MST)=6 days; FIG. 1). Additionally, to determine whether the absence of the disease upon infection with the avirulent strain was due to the inability of the virus to penetrate the central nervous system, animals were infected intracranially with 1000 PFU of virus as opposed to control animals that were injected with medium alone intracranially. It was observed that the animals did not develop any sign of illness and mortality after infection.

Furthermore, when the mice were infected intracranially with a higher dose of the EEEV strains (10E6 PFU) to determine whether an increase in virus dose could change the outcome of the infection, none of the mice infected with higher dose of BeAr 436087 succumbed to infection as opposed to mice infected with other EEEV strains that succumbed quickly to the disease (AST=2-3 days; FIG. 2), thereby suggesting that BeAr 436087 strain was unable to replicate efficiently in the brain or to cause severe pathology in the animals. Subsequently, the survivors were infected with a more virulent strain of EEEV either one month or three months post-infection and no mortality was observed upon challenge, thereby demonstrating that the avirulent strain was able to induce long-lasting immunity against EEEV. Additionally, to determine whether the avirulent strain was also unable to cause neurological disease in hamsters, the avirulent strain was also compared in hamsters that were 5-7 and 12 week old. It was observed that 5-7 week old hamsters developed neurological disease whereas the 12 week old hamsters survived the infection with the attenuated strains. In all cases, hamsters infected with more virulent EEEV strains succumbed to infection.

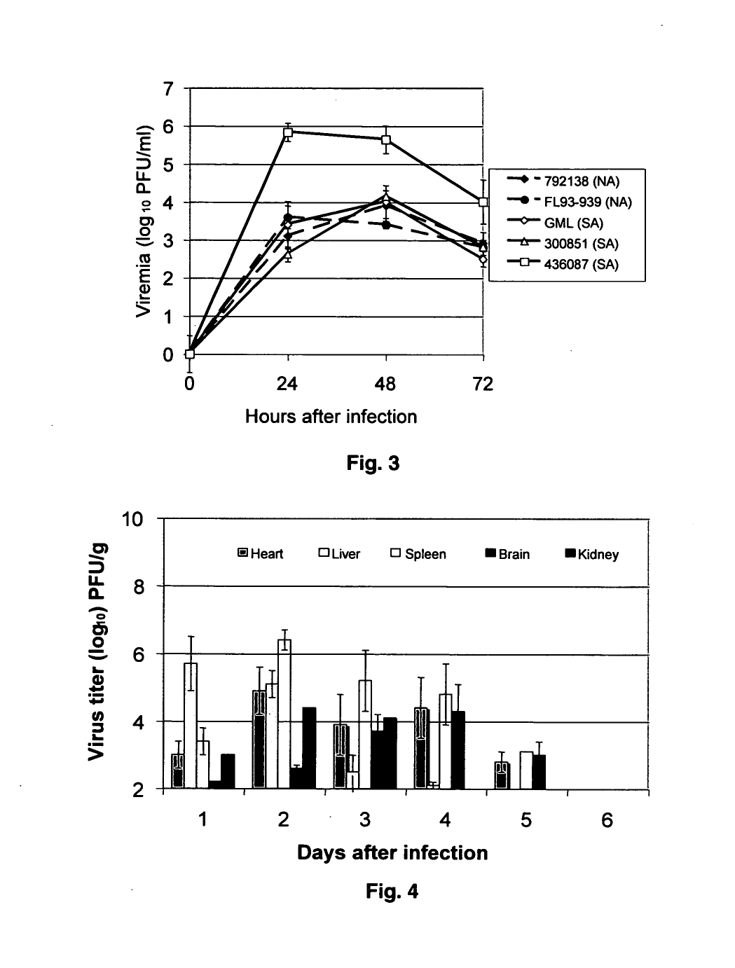

In order to examine the replication of virus in vivo, 4 animals infected subcutaneously and 3 infected intracranially were sacrificed daily and their brain, heart, lung, liver and kidney homogenized and titrated in Vero cells. Serum samples were also obtained daily from the mice to determine the viremia levels in the infected animals. It was observed that the viremia in animals infected with the avirulent strain was about 10-fold higher than the viremia in animals infected with the other EEEV strains used in the study (FIG. 3).

Analysis of the replication of the virus in the brain, heart, spleen, kidney and liver demonstrated that mice infected with the avirulent strain cleared the virus from the brain by day 6 post-infection as opposed to mice infected with more virulent strains (FIG. 4), in which replication in the brain continued to increase until the time of encephalitic death (FIG. 5). The clearance of the avirulent strain from the circulation and from all organs appeared to correlate with the appearance of neutralizing antibodies, which did not differ among the virulent and avirulent strains.

Furthermore, histopathological studies revealed lesions only on days 6-7 post-infection in mice infected with the avirulent strain. For instance, a mild focal meningoencephalitis was observed in the white matter of the cerebellum in 3 of the 4 animals infected with the avirulent strain; a small foci of perivascular cuffing was observed in the olfactory bulb in one of the mice infected with the avirulent strain; a mild hepatitis with lobular, interstitial inflammation and microvesicular steatosis was observed in the liver on day 6 PI and a focal interstitial inflammation was observed in the kidney on day 6 PI.

As opposed to these mild changes observed in mice infected with the avirulent strain, mice infected with the virulent EEEV strains developed disseminated, meningoencephalitis, associated with microglial activation, perivascular cuffing and mononuclear cell infiltration from days 4-7. The pathological manifestation in the liver was severe and mainly characterized by diffuse hepatocellular necrosis, interstitial hepatitis, congestion, diffuse microhemorrhages and infiltration of mononuclear cells.

Immunohistochemical analysis also confirmed the presence of small foci of viral antigen in the neurons of mice infected with the avirulent strain beginning on day 3 PI. The number of positive cells remained approximately constant until day 6 PI. Moreover, the number of viral antigen positive cells was significantly lower compared to the number of positive cells detected in the brain of mice infected with the virulent strain, which increased significantly throughout the course of infection. This result suggested that the avirulent strain replicated in neurons but was unable to spread efficiently to neurons or cause disseminated encephalitis. As opposed to this, the virulent strains replicated in the neurons, rapidly disseminated within the brain causing acute, disseminated encephalitis and death of the animals.

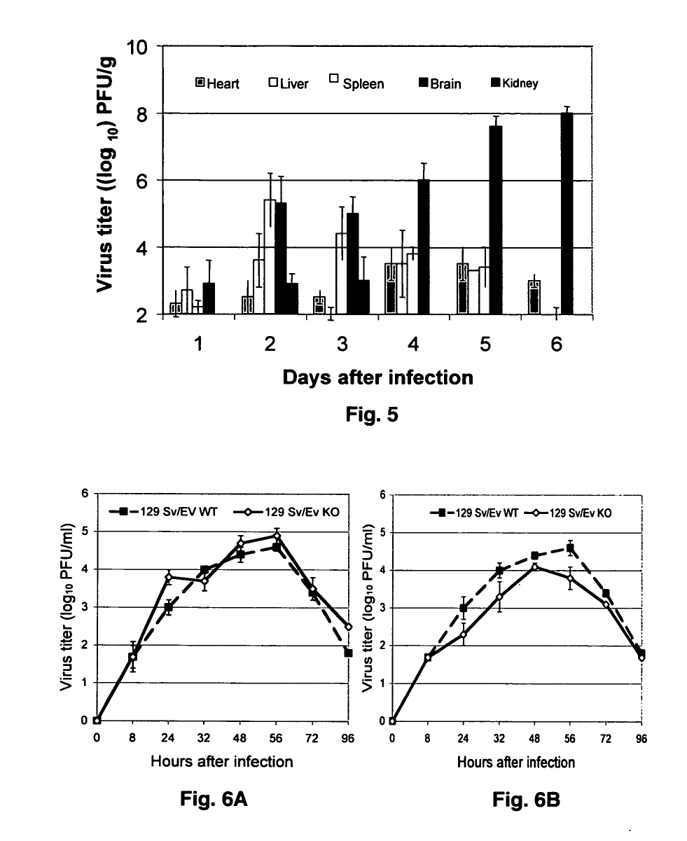

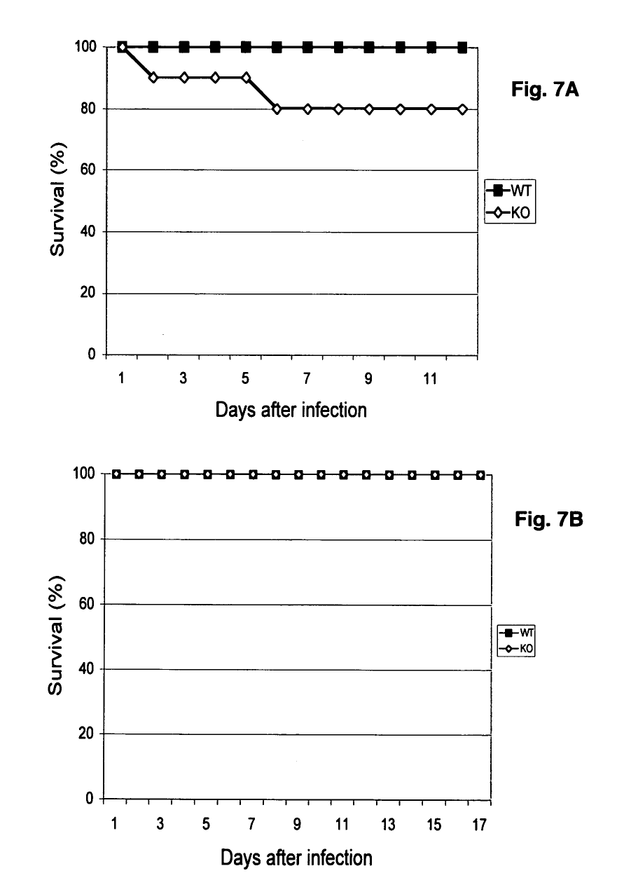

Since there was a difference in virulence that was observed with the avirulent strain, whether this difference was due to resistance to Type I and/or Type II IFN was also examined. As described earlier the IFN α/β receptor −/−, IFN γ receptor −/− and wild type mice were infected subcutaneously with EEEV strains 792138 and BeAr 436087 and the viremia and mortality were recorded. The viremia levels in IFN α/β and γ receptor −/− versus wild type control mice is shown in FIGS. 6A-B, respectively. Although two KO mice succumbed to infection, no significant difference in viremia or mortality was observed in the IFN-α/β KO mice compared to the wild type control group infected with the avirulent strain (FIG. 7A). No mortality was observed in the IFN γ receptor KO mice group and wild type group infected with the avirulent strain (FIG. 7B). These results suggested that the attenuation of the virulent strain was Type I and Type II IFN-independent. In distinct contrast, IFN-α/β KO mice infected with the strain 792138 had a significant difference in viremia and mortality compared to wild type mice, thereby demonstrating the importance of Type I IFN in protection against this strain. No difference in viremia or mortality was observed between IFN-γ deficient and wild type mice infected with this strain. Furthermore, in order to identify possible amino acids that could be responsible for the difference in the virulence among the strains, genomic sequences of the avirulent strain and three other EEEV strains were determined and compared. A large number of nucleotide and amino acid differences throughout the genome were observed. The highest degree of amino acid divergence was observed in the nsP3 region followed by the E2 glycoprotein. Table 3 summarizes the results of the sequence comparison between the strains FL93-939 and the avirulent strain BeAr436087.

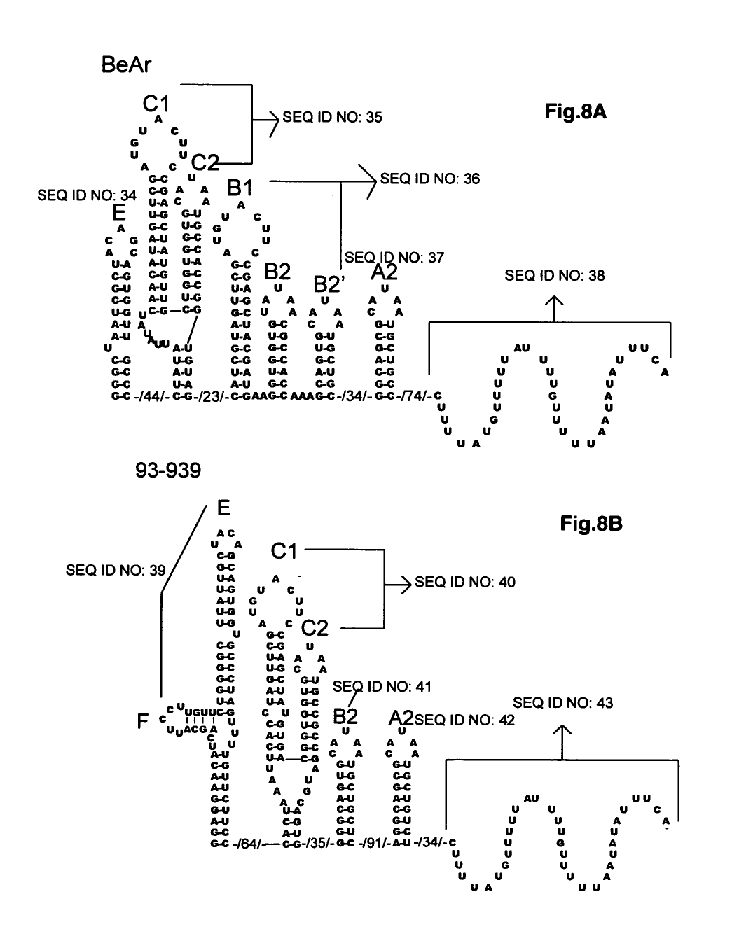

Identification of possible amino acids and/or genes that could be associated with the difference in mouse virulence were difficult to determine due to high degree of amino acid differences in the structural and nonstructural genes. However, when the predicted secondary structures of the 5′ and 3′ end of virulent and avirulent EEEV strains were compared to determine the potential effects of the differences in repeated elements, it was observed that the avirulent strain possessed 5 extra repeated elements that form two extra hairpin loops structures at the 3′ end compared to the NA strains (FIGS. 8A-B).

TABLE 3
Nucleotide and amino acids differences for BeAr436087
compared to virulent strain FL93-939.

| Virus | % nucleotide | % Amino acid |
|---|---|---|
| genes | differences | differences |
| nsP1 | 11.6% | 7.8% |
| nsP2 | 24.2% | 8.5% |
| nsP3 | 28.9% | 24.8% |
| | 2.5% * | 2.5% ** |
| nsP4 | 22.7% | 7% |
| C | 21.5% | 4.2% |
| E3 | 20.5% | 9.8% |
| E2 | 24.2% | 16% |
| 6K | 19% | 9% |
| E1 | 22.8% | 9.5% |

* indicates nucleotide deletions.
** indicates amino acid deletions.

Next, the structural and non-structural genes of the avirulent and virulent strain of EEEV were examined as described in subsequent examples to determine if these genes were responsible for the difference in virulence.

### Example 10

### Viruses

The virus strains BeAr 436087 and FL93-939 were provided by the University of Texas Medical Branch World Reference Center for Emerging Viruses and Arboviruses. Strain BeAr 436087 was isolated from a mosquito pool in Fortaleza, Brazil and passaged twice in suckling mouse brains to generate RNA for this study. Strain FL 93-939 was also isolated in Vero cells from a pool of Culiseta melanura mosquitoes and passage once in Vero cells and once in a suckling mouse brain to generate RNA. For the suckling mouse passages, 2-3 day-old mice were inoculated intracranially with each virus strain and a 10% suspension of homogenized brain tissue was prepared after morbidity or mortality was observed. The titers of the virus stocks was determined by plaque assay in Vero cells (Wang et al. 1999).

### Example 11

### RNA Extraction and RT-PCR

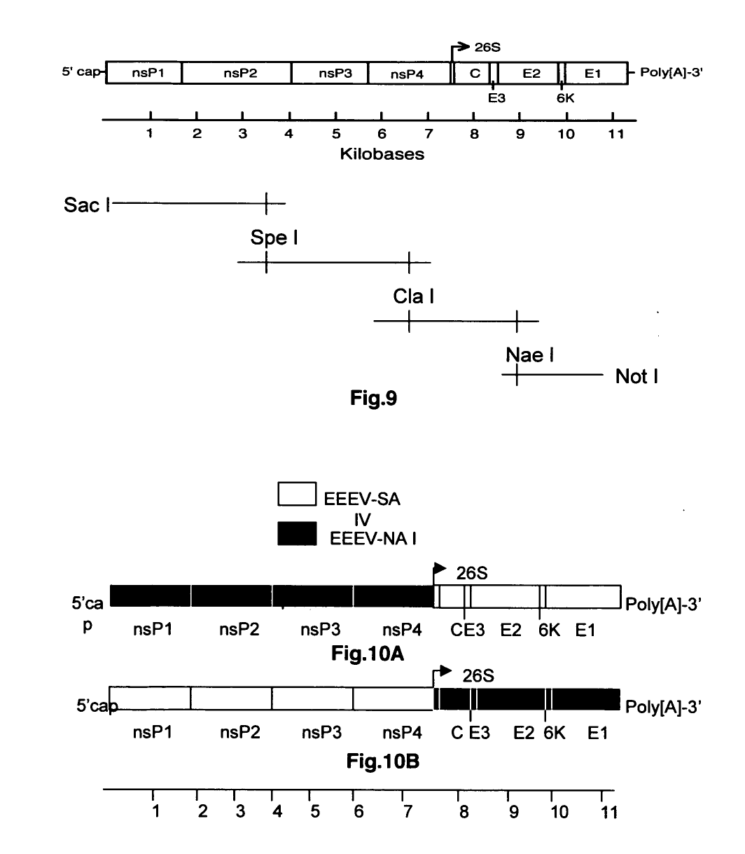

RNA extraction and RT-PCR was performed as described supra. The genome of FL 93-939 was divided into 5 overlapping fragments spanning appropriate unique restriction sites as shown in FIG. 9 for the amplification.

### Example 12

### Construction of Infectious Clones

To generate the pM1 EEEV-FL93-939 (NA) infectious clone, the low copy-ampicillin resistant plasmid pM1 vector was used for the final construction. Fragments were sequentially cloned using appropriate unique restriction sites. Each cloning step was confirmed by restriction digestion and sequence analysis of the junctions to ensure no aberrant or lethal mutations were introduced during the cloning process.

### Example 13

### Construction of Chimeric Viruses

To construct the first chimeric infectious clone, pM1-EEEV-NA/SA, a subclone covering the entire structural gene region of the strain BeAr 436087 (SA) was created. To facilitate the interaction between the 5′ end and the 3′ end of the genome for viral RNA synthesis, the 3′end of the strain BeAr 436087 (SA) was exchanged for the 3′end of the FL93-939 (NA) strain in the subgenomic clone. Two PCR products were generated using chimeric primers: a) PCR-1 using primers EEE-SA-11,157 (CCACAAGCTTACCAGCGTAGTCACCTGC; SEQ ID NO: 26) and EEE-SA(E1)/NA(3′)-R (TATGTGGTTGACAAGATGTTAGTGTTTGTGGGTGA; SEQ ID NO: 27) and b) PCR-2 using primers EEE-SA(E1)/NA(3′)-F (TCACCCACAAACACTAACATCTTGTCAACCACATA; SEQ ID NO: 28) and pGEM-R (ACTCAAGCTATGCATCCAACGCGTTGGGA; SEQ ID NO: 29). A third PCR amplification was performed with primers EEE-SA(E1)/NA(3′)-F and pGEM-R using as template PCR 1 and 2 products in the same reaction. The resultant PCR product of about 700 bp was subcloned into the pGEM vector. The fragment containing the 26S and the exchanged 3′end was replaced in the NA infectious clone using Sfi I/Not I restriction enzymes. The restriction site for the Sfi I enzyme was located few nucleotides downstream of the capsid coding region; however, the beginning of the capsid region was highly conserved between the two strains and therefore no amino acid change within the capsid protein was introduced into the final chimera construct. FIG. 10A illustrates the genetic organization of the pM1 NA/SA chimera.

To construct the second chimera pM1-EEEV-SA/NA, the subclone covering the entire structural gene region of the strain FL93-939 (NA), generated during the construction of the pM1-EEEV-FL93-939 (NA) infectious clone was used. A similar strategy was used to exchange the 3′end of the strain FL93-939 (NA) for the 3′end of the BeAr436087 (SA) strain in the subgenomic clone. Two PCR products were generated: a) PCR-1 using primers EEE-NA-11,068 (CCACAAGCTTCACTGCAAACATCCATC; SEQ ID NO: 30) and EEE-NA(E1)/SA(3′)-R (GGTAATTTACTGCTAGTATTAATGTCTATGGAAGA; SEQ ID NO: 31) and b) PCR-2 using primers EEE-NA(E1)/SA(3′)-F (TCTTCCATAGACATTAATACTAGCAGTAAATTACC; SEQ ID NO: 32) and pGEM-R (ACTCAAGCTATGCATCCAACGCGTTGGGA; SEQ ID NO: 33). A third PCR amplification was performed with primers EEE-NA(E1)/SA(3′)-F and pGEM-R using as template PCR 1 and 2 products in the same reaction. The resultant PCR product of about 700 bp was subcloned into the pGEM vector. The final plasmid containing the 26S and the exchanged 3′end was engineered into the plasmid harboring the nonstructural genes of the SA strain by Sfi I/Not I digestion. FIG. 10B illustrates the genetic organization of the pM1 SA/NA chimera construct. In all cases, the fragments were joined in a single ligation reaction using the T4 DNA ligase (Invitrogen, Carlsbad, Calif.).

### Example 14

### RNA Transcription and Transfection

Plasmids were purified by using the Maxiprep (Qiagen, Valencia, Calif.) and linearized with restriction endonuclease NotI to produce cDNA templates for RNA synthesis. In vitro transcription was performed as previously described (Anishchenko 2004) using the T7 RNA polymerase promoter and the m⁷G(5′)ppp(5′)G RNA cap structure analog (New England Biolabs, Beverly, Mass.). RNA was transfected into BHK-21 cells by electroporation as previously described (Anishchenko 2004; Powers 1996) and the virus was harvested 24 hr after transfection.

### Example 15

### Plaque Assays

Plaque assays were performed as described (Powers 2000) using Vero cells. Briefly, cells were seeded into six-well tissue culture plates and allowed to grow to confluency. Tenfold dilutions of the virus were adsorbed to the monolayers for 1 h at 37° C. A 3-ml overlay consisting of minimum essential medium with 0.4% agarose was added and the cells were incubated at 37° C. for 48 hr. Agar plugs were removed, and the cells were stained with 0.25% crystal violet in 20% methanol.

### Example 16

### Virus Replication In Vitro

Vero and C710 cells were seeded into 12-well plates and two days later infected with parental, infectious clone viruses and chimeric viruses at a multiplicity of infection of 10. Briefly, medium was removed from the cells and viruses were allowed to adsorb for 1 hr at 37° C. After the incubation, the cells were washed twice with saline solution and fresh medium was then added to the cells. Supernatant fluids were collected at 0, 8, 24, 32 and 48 hr after infection and titrated by plaque assay.

### Example 17

### Mouse Virulence Studies

Viruses rescued from the infectious clones and parental viruses were inoculated into five 5-7 week-old mice (Harlan Laboratories, Indianapolis, Ind.) for viremia and mortality comparison. Mice were bled 24, 48, and 72 hrs and the sera were assayed by plaque assay. Chimeric viruses were inoculated subcutaneously into ten 5-7 week-old mice with 1000 PFU of virus. Similarly, mice were bled 24, 48, 72 hrs and the sera assayed by plaque assay. All animals were monitored daily for clinical signs of disease including fever, lethargy, paralysis or death.

### Example 18

### Statistical Analysis

Statistical comparisons were performed using the paired Student's T test and one-way ANOVA with Dunn's multiple comparison test to determine if the differences among samples of all groups were significant. Survival curves were analyzed using the logrank test included in the GraphPad Prism program (San Diego, Calif.). Values of p≦0.05 were considered significant.

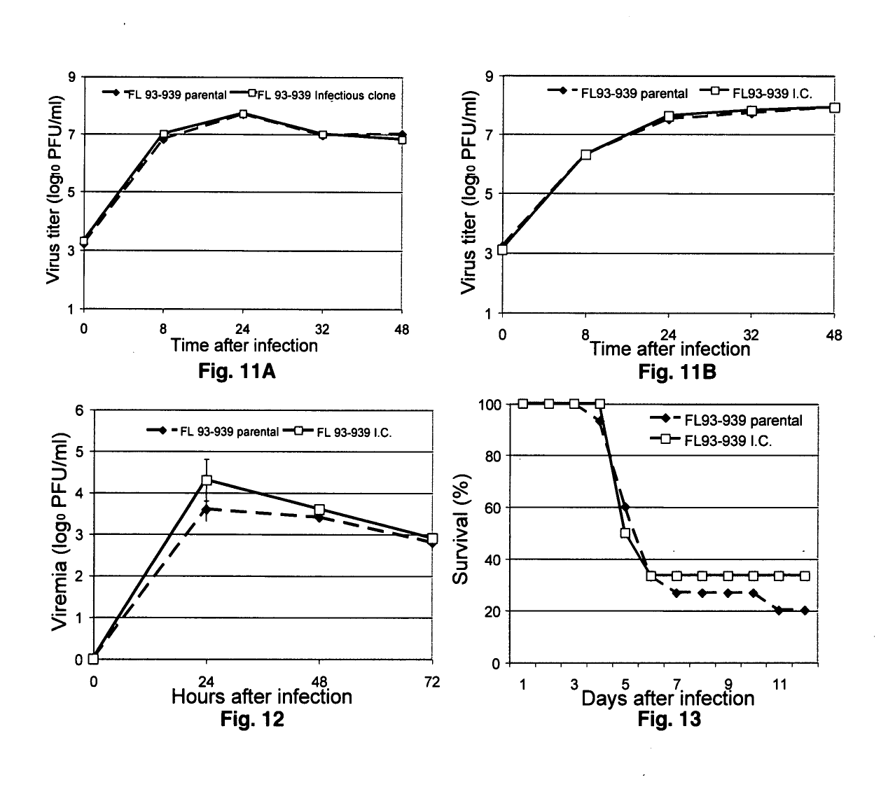

When the replication kinetics were compared, no significant difference in virus replication was observed between the pM1-EEEV-FL93-939 (NA) infectious clone and the parental virus in either Vero or C710 mosquito cell line (p<0.05) (FIGS. 11A-B). Vero cells infected with both parental and rescued virus from the infectious clone produced a visible cytopatic effect (CPE) as early as 24 hr PI. The CPE in C710 infected with both parental and infectious clone virus was more visible at 48 hr PI. These results indicated that viruses rescued from the infectious clone and its parent were nearly identical in vitro.

Next, when the replication of both parental and infectious clone viruses were compared in vivo, it was observed that all animals developed clinical signs of disease beginning on day 3 PI (FIG. 12). Animals became lethargic, anorexic and ruffling of the hair was evident, which was consistent with previously published observations for experimental mouse infections with EEEV (Vogel et al. 2005). By day 4 PI, mice developed more evidence of CNS involvement and posterior limb paralysis was observed in the majority of the animals. For the survival data, euthanization of the animals was treated as mortality.

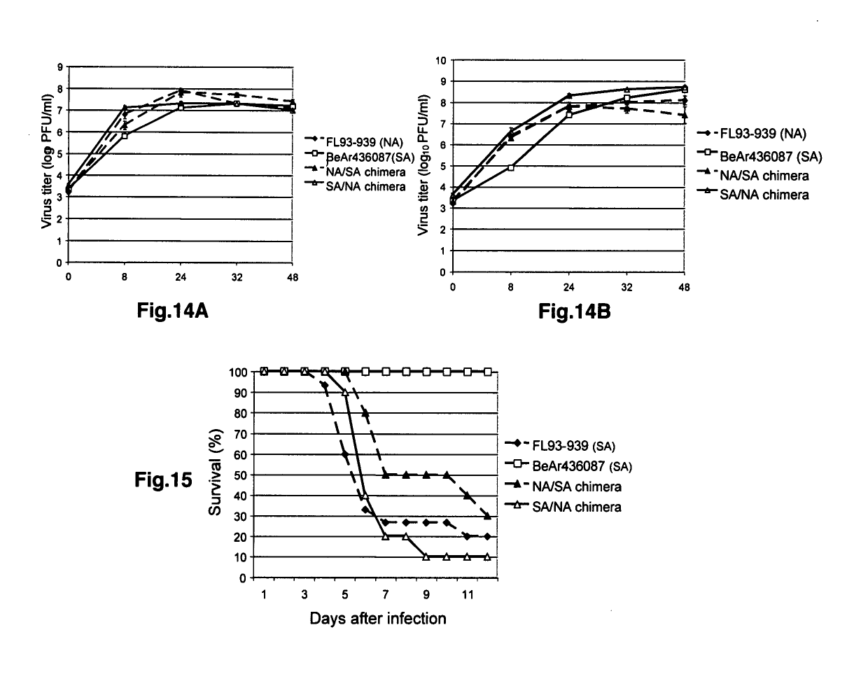

Furthermore, the replication of the NA/SA and SA/NA chimeras was also analyzed and compared to the replication of parental viruses in Vero and C710 mosquito cells. At 8 hr PI in Vero cells, replication levels of the chimeric viruses were intermediate between the parental viruses (P<0.05). However, at 24 hr PI, replication of the SA/NA chimera was more similar to that of the SA strain (P>0.05) than to that of the NA strain (P<0.05). Similarly, replication of the NA/SA chimeric virus was similar to that of replication of NA strain (P>0.05) and differed statistically from the SA/NA chimera and SA strain (P<0.05). At 32 hr and 48 hr PI, no significant difference was observed among parental and chimeric viruses (P>0.05) (FIG. 14A).

The replication of the chimera and parental viruses in C710 mosquito cells showed some differences. At 8 hr and 24 hr PI, the replication of the SA strain was about 12 and 4-9 fold lower than both chimeras and the NA strain, respectively. In contrast, replication of the chimeras and the NA strain did not differ (P>0.05). No significant differences in virus replication were observed among the viruses after 24 hr PI (FIG. 14B).

As discussed herein, since the SA strain BeAr436087 was observed to be avirulent in mice and the NA strain FL-93-939 caused 80% mortality in mice, mouse virulence phenotype of both the chimeras were examined. Both viruses derived from the chimeras resulted in paralysis and death in mice within 4-7 days as did the parental NA virus. No statistically significant difference in mortality was observed among the chimeras and the NA virus (FIG. 15) (P>0.05), although the NA/SA chimera resulted in a mean survival time (MST) that was 3 days longer than that of the NA strain and the SA/NA chimera in mice.

Serum viremias were determined for mice infected with both chimeras and parental viruses. The viremia for the NA/SA strain was comparable to the viremia of the NA parental strain and reached 3.6-3.9 log₁₀ PFU/ml at 24 hr PI. Interestingly, viral titers for the SA/NA chimera were similar to the SA parental virus. Both SA/NA chimera and the SA parental virus induced more than 10 fold higher viremia (5.3 log₁₀ PFU/ml) in the mice when compared to the NA strain and the reciprocal chimera (P<0.05).

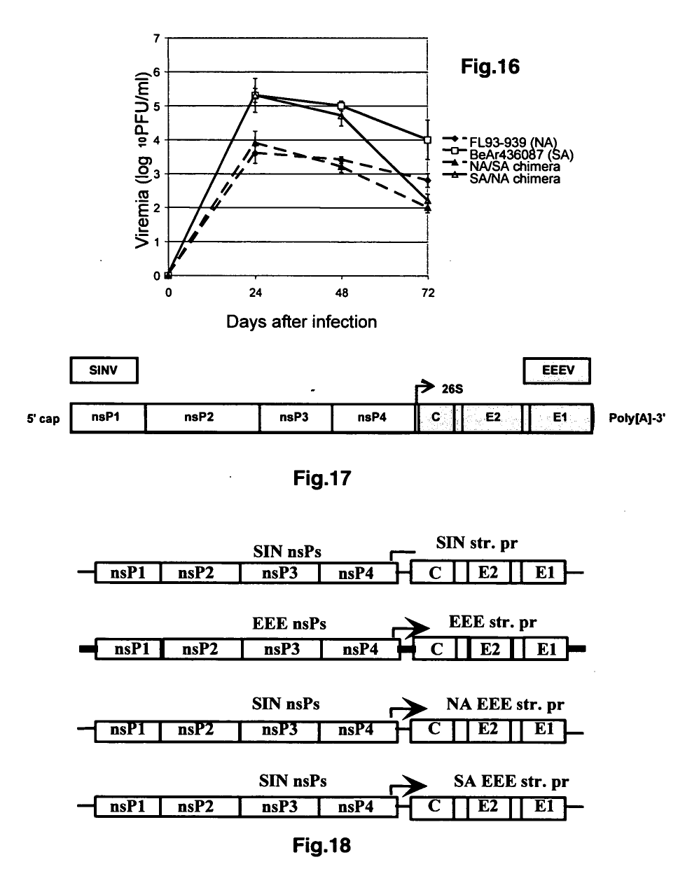

The results of this study demonstrated once more that viremia levels do not correlate with neurovirulence, since the avirulent strain BeAr436087 induced more than 10 fold higher viremia than the other strains of EEEV analyzed. Similarly, both NA/SA and SA/NA chimeras differed by 14-fold in viremia levels, yet these viruses exhibited the same virulence phenotype for mice (FIG. 16).

### Example 19

### Construction of Sindbis-EEE Chimeric Viruses

In order to construct Sindbis-EEE chimeric viruses (FIG. 17), the cis-acting RNA elements of the recombinant genome that are required for replication and transcription of the subgenomic RNA (5′ untranslated region (UTR), 3′ UTR and the 26S promoter) were derived from SINV. Additionally, all the genes of the nonstructural proteins were SINV-specific as well. The structural genes were acquired from the various EEEV strains. The examples of EEEV strains that could be used to construct such viruses are 792138, FL93-939, GML903836, BeAr 300851 and BeAr436087. This strategy of virus design enabled maintenance of optimal combinations of factors essential i) for RNA replication, including replicative enzymes and recognized RNA sequences, and ii) factors required for efficient translation of the subgenomic RNA including the sequence and secondary structure of the 26S 5′UTR.

The promoter element located upstream of the subgenomic RNA transcription start and the four 5′ terminal nucleotides of the subgenomic RNA were made SINV specific since they represented the end of nsP4 and the termination codon of the nsP-coding open reading frame (ORF). An additional C→→T mutation was introduced at position 24 of the 26S 5′UTR to compensate for the mutation G→→A at position 4 and to maintain the computer-predicted 5′ terminal secondary structure of the chimeric virus close to that of EEEV subgenomic RNA.

The immunogenicity of Sindbis-EEE chimeric viruses in 4-week old female NIH-Swiss mice and 4-week old female golden Syrian hamsters was determined using strain 339 (described supra) and strain 464. The strain 464 comprised of the structural genes of FL93-939 strain of EEEV in the Sindbis virus strain Toto1101 genome backbone. The vaccinations of these animals was carried out as follows: 8 mice were vaccinated subcutaneously with 5×10E7 plaque forming units of strain 339; 3 hamsters were vaccinated subcutaneously with 5×10E7 plaque forming units of strain 339; 8 mice were vaccinated subcutaneously with 5×10E7 plaque forming units of EEEV strain FL93-939; 3 hamsters were vaccinated with 5×10E7 plaque forming units of EEEV strain FL93-939; 5 mice and 3 hamsters were sham-vaccinated with PBS and 8 mice and 3 hamsters were not vaccinated. All the animals were bled on day 21 post-vaccination and plaque reduction neutralization tests were performed with EEEV strain FL93-939. Table 4 shows the antibody titers in animals 21 days post-vaccination.

TABLE 4
Antibody titers in animals 21 days post vaccination.

| | Vaccine | Mean antibody | Standard |
|---|---|---|---|
| Animal | strain | titer | deviation |
| mouse | 339 | 72 | 42 |
| hamster | 339 | 53 | 23 |
| mouse | 464 | 71 | 46 |
| hamster | 464 | 60 | 35 |
| mouse | sham | <20 | |
| hamster | sham | <20 | |

### Example 20

### Efficacy of the SIN/EEE Vaccine in Mice

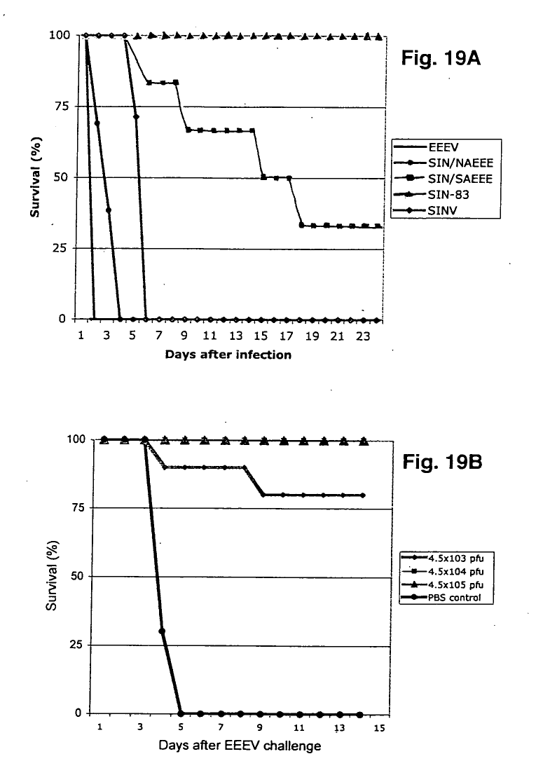

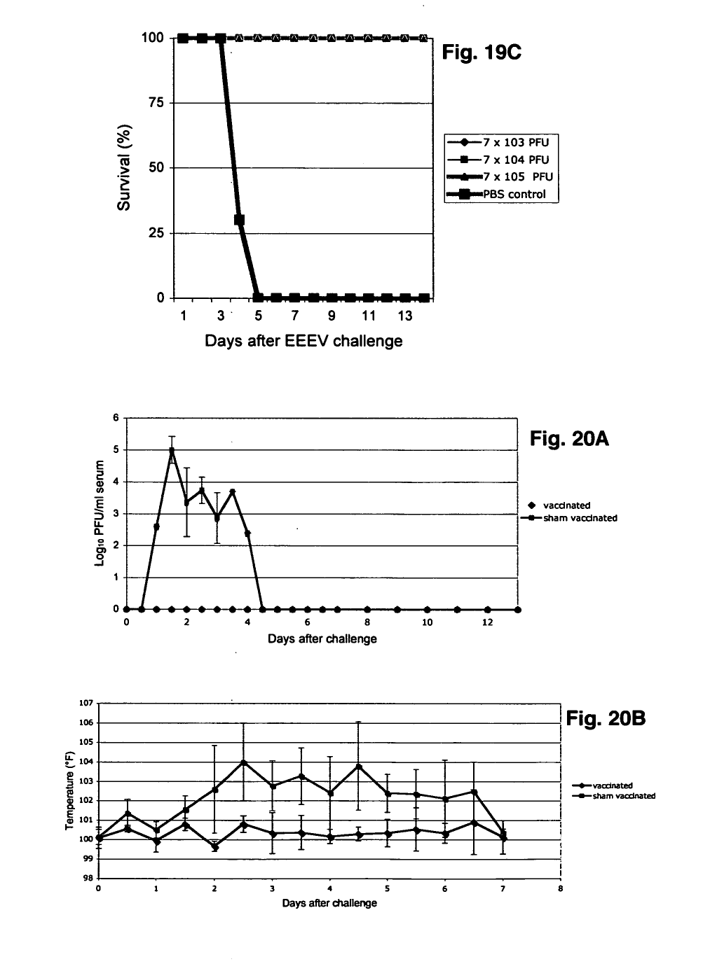

The DNA encoding SIN/EEE chimeras that were used herein are as shown in FIG. 18. The chimera comprising the North American EEE strain caused 70% mortality in humans whereas the chimera comprising the South American EEE strain caused no mortality in human. The attenuation of the Sindbis-EEE chimeric viruses was examined in a severe challenge mode. Briefly, 6-day old Swiss NIH mice were injected intracranially with 10 E6 PFU of the Sindbis virus containing either the North or South American EEE virus structural genes as well as the wild type Sindbis (Ar339) or EEE virus (FL93-939) or the SIN-83 Sindbis-VEE virus. It was observed that both the chimeric Sindbis-EEE virus strains were attenuated compared to the wild type EEE virus with longer average survival (FIG. 19A). However, the chimera with the South American EEE virus structural genes was more attenuated than the Sindbis-EEE virus with the North American EEE virus structural genes.

The efficacy of the Sindbis-EEE chimeric viruses were then examined in the murine model. Briefly, cohorts of ten 6 week old NIH Swiss mice were vaccinated with 3 different doses of the Sindbis-EEE virus (North American EEE virus structural genes) and challenged intraperitoneally 4 weeks later with 10E6 PFU of EEE virus (North American strain FL93-939). All but the lowest dose (4.5 10E3) protected all mice against lethal challenge, while all of the sham-vaccinated animals developed lethal encephalitis (FIG. 19B). Additionally, in order to examine the efficacy of the chimera comprising South American EEEV structural genes, cohorts of ten 6-week old NIH Swiss mice were vaccinated with 3 different doses of the Sindbis-EEE virus (South American EEE virus structural genes) and challenged intraperitoneally 4 weeks later with 10E6 PFU of EEE virus (North American strain FL93-939). All doses including the lowest (7 10E3) protected all mice against the lethal challenge, while all of the sham-vaccinated animals developed lethal encephalitis (FIG. 19C).

Furthermore, to assess the immunogenicity of the Sindbis-EEE virus in adult (6-week old mice), cohorts of 5 animals were vaccinated with a range of doses and the serum neutralizing antibody levels were assessed 4 weeks later. All animals developed mean antibody titers ranging from 120-777 (reciprocal dilutions yielding 80% plaque reduction). In distinct contrast, the sham vaccinated mice failed to develop detectable antibodies (Table 5).

TABLE 5
Mean plaque reduction neutralization antibody titers
in mice after vaccination with different doses of
the Sindbis-EEE virus (North American strain).

| Vaccine dose (Log₁₀ | PRNT titer | PRNT titer |
|---|---|---|
| PFU) | EEEV-NA ± SD | EEEV-SA ± SD |
| 3.0 | 128 ± 44 | 20(3/5) |
| 3.9 | 120 ± 57 | <20 |
| 4.0 | 320 ± 196 | 20 (3/5) |
| 4.9 | 132 ± 39 | <20 |
| 5.0 | 533 ± 165 | 20 ± 0 |
| 5.9 | 777 ± 337 | 80 ± 57 |

### Example 21

### Immunogenicity of the Sindbis-EEE Chimeric Viruses in Horses

The immunogenicity of the Sindbis-EEE chimeric viruses in horse was determined using strain 339. This strain comprised of the structural genes from North American strains FL93-939 in the Sindbis virus strain TR339 genome backbone. Mares that were 1-2 year old and alphavirus PRNT antibody negative were vaccinated subcutaneously with 10E3, 10E5 or 10E7 PFU. All animals were bled weekly for 4 weeks post-vaccination and plaque reduction neutralization tests were performed with EEEV strain NJ60. Table 6 shows the antibody titers in the horses that were vaccinated with the chimeric virus on days 7, 14, 21 and 28 after vaccination.

TABLE 6
Antibody titers

| Horse | Age | Dose | Days after vaccination | | | |
|---|---|---|---|---|---|---|
| No. | (yrs) | (log₁₀PFU) | 7 | 14 | 21 | 28 |
| SW57 | 1 | 3 | <10 | <10 | <10 | <10 |
| SW59 | 2 | 5 | 20 | 1280 | 640 | 640 |
| SW58 | 1 | 7 | <10 | >2560 | >2560 | >2560 |

None of the horses developed any clinical signs of illness or a febrile response after vaccination. Additionally, SIN-EEE virus was not detected in the blood sampled on days 1-5.

To determine the efficacy of the chimeric viruses described supra cohort of five 1-2 year old horses were vaccinated with 10E5 PFU of the Sindbis-EEE chimeric virus and challenged 28 days later with strain FL93-939 of North American EEE virus. Sham-vaccinated horses were used as negative controls. None of the vaccinated animals developed detectable disease, viremia (FIG. 20A) or a febrile response (FIG. 20B) after vaccination. In distinct contrast, all of the sham-vaccinated horses developed clinical encephalitis with high fever and viremia observed in most (Table 7).

TABLE 7
Summary of SIN/EEE efficacy in horses

| | | Number with | |
|---|---|---|---|
| | | viremia (>10 | Number with |
| | Number with | PFU/ml) in | febrile |
| Treatment | encephalitis | serum | response >101° F. |
| Sham | 5/5 | 3/5 | 5/5 |
| vaccination | 0/5 | 0/5 | 1/5 |
| SIN/EEE 10⁵ | | | |
| PFU | | | |

The following references were cited herein:
Ahola et al., J Virol, 2000, 74 (15):6725-6733.
Alice, Boletim Instituto Biologico da Bahia, 1956, 3: 3-9.
Anishchenko et al., J Virol, 2004, 78 (1):1-8.
Bernard et al., Virology, 2000, 276 (1):93-103.
Brault et al., J Virol, 2002, 76 (4):1718-1730.
Brault et al., Proc Natl Acad Sci USA, 2004, 101 (31): 11344-11349.
Brown et al., J Gen Virol, 1975, 27: 111-116.
Brynes & Griffin, J Virol, 1998, 72 (9): 7349-7356.
Calisher and Karabatsos, Arbovirus serogroups: definition and geographic distribution. In: MTP, editor, translator and editor The Arboviruses: Epidemiology and Ecology, Vol. I. Boca raton, FL: CRC Press, 1988: p. 19-57.
Calisher et al., Intervirology, 1980, 14 (5-6):229-232.
Casals, J Exp Med, 1964, 119:547-565.
Causey and Theiler, Am J Trop Med Hyg, 1958, 7 (1):36-41.
Causey et al., Revista Servico Especial Saude Publica, 1962, 12:47-50.
Charles et al., Virology, 1995, 208 (2):662-671.
Davis et al., Virology, 1991, 183 (1):20-31.
Davis et al., Proc Natl Acad Sci USA, 1986, 83 (18):6771-6775.
Dremov et al., Acta Virol , 1978, 22:139-145.
Dropulic et al., J Virol, 1997, 71 (8):6100-6105.
Fazakerley et al., Virology, 1993, 195 (2):627-637.
Fothergill et al., N Engl J Med, 1938a, 219:41.
Frolova et al., J Virol, 2002, 76 (22):11254-11264.
Glitner and Shahan, N Amer Vet, 1933, 14:25-27.
Grieder et al., Virology, 1995, 206 (2):994-1006.
Grimstad, Adv Virus Res, 1983, 28:357-438.
Heil et al., J Virol, 2001, 75 (14):6303-6309.
Klimstra et al., J Virol, 1998, 72 (9):7357-7366.
Lustig et al., J Virol, 1988, 62 (7):2329-2336.
Polo & Johnston, J Virol, 1990, 64 (9):4438-4444.
Powers et al., Proc Natl Acad Sci., 1996, 93:4187-4191.
Powers et al., J Virol, 2000, 74 (9):4258-4263.
Sabattini et al., Historical, epidemiological and ecological aspects of arboviruses in Argentina: Togaviridae, alphavirus. An overview of arbovirology in Brazil and neighboring countries. Instituto Evandro Chagas, Belem, 1998
Scott and Weaver, Adv Virus Res., 1989, 37:277-328.
Sharpe et al., Nature, 1990, 346 (6280):181-183.
Shope, Am J Epidemiol, 1966, 84 (3):467-477.
Solyanik et al., Genetika, 1972, 8:109-114.
Spotts et al., J Virol, 1998, 72 (12):10286-10291.
Strauss & Strauss, Microbiol Rev, 1994a, 58 (3):491-562.
Swoveland and Johnson, Virology, 1989, 170 (1):131-138.
TenBroeck and Merrill, Proc Soc Exp Biol Med, 1933, 31:217-220.
Travassos da Rosa, Arboviruses isolated in the Evandro Chagas Institute, including some described for the first time in the Brazilian. Instituto Evandro Chagas, Belem, 1998
Tucker & Griffin, J Virol, 1991, 65 (3):1551-1557.
Tucker et al., J Virol, 1993, 67 (8):4605-4610.
Tuittila & Hinkkanen, J Gen Virol, 2003, 84 (pt 6):1525-1533.
Tuittila et al., J Virol, 2000, 74 (10):4579-4589.
Vasconcelos and Travassos da Rosa, Arboviruses pathogenic for man in Brazil. An overview of arbovirology in Brazil and neighboring countries. Instituto Evandro Chagas, Belem, 1998
Vogel et al., Am J Pathol, 2005, 166 (1):159-171.
Walder et al., Arboviruses in Mediterranean countries. In: J Vesenjak-Hirjan, editor, translator and editor: Fisher, Stuttgart, 1980, p. 237-250.
Wang et al., J Virol, 1999, 73 (5):4266-4271.
Weaver et al., Arch Virol Suppl, 2004, 18:43-64.
White et al, J Virol, 2001, 75 (8):3706-3718.
Zuker, Nucleic Acids Res, 2003, 31:1-10.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

1. An attenuated Eastern equine encephalitis virus comprising:
a DNA encoding a chimeric Eastern equine encephalitis virus, said DNA comprising a Sindbis virus cDNA fragment and an Eastern equine encephalitis virus cDNA fragment comprising structural protein genes.

2. The attenuated Eastern equine encephalitis virus of claim 1, wherein said Sindbis virus cDNA fragment comprises cis-acting sequences from 5′ and 3′ termini, 26S promoter and nonstructural protein genes.

3. (canceled)

4. The attenuated Eastern equine encephalitis virus of claim 3, wherein said Eastern equine encephalitis virus cDNA fragment is derived from FL93-939 or BeAr436087 strain of Eastern equine encephalitis virus.

5. The attenuated Eastern equine encephalitis virus of claim 1, wherein the chimeric Eastern equine encephalitis virus has protein content identical to wild-type Eastern equine encephalitis virus.

6. A vector comprising a DNA encoding a chimeric Eastern equine encephalitis virus, said DNA comprising a Sindbis virus cDNA fragment and a Eastern equine encephalitis virus cDNA fragment comprising structural protein genes.

7. A host cell comprising and expressing the vector of claim 6.

8. (canceled)

9. A pharmaceutical composition comprising the attenuated Eastern equine encephalitis virus of claim 1 and a pharmaceutically acceptable carrier.

10. An immunogenic composition comprising a live attenuated Eastern equine encephalitis virus vaccine, wherein said vaccine comprises the attenuated Eastern equine encephalitis virus of claim 1.

11. An immunogenic composition comprising an inactivated Eastern equine encephalitis virus vaccine, wherein said vaccine comprises the attenuated Eastern equine encephalitis virus of claim 1, wherein said attenuated Eastern equine encephalitis virus is inactivated.

12. A method of protecting an individual from infections resulting from exposure to Eastern equine encephalitis virus, comprising administering pharmaceutically effective amount of the immunogenic composition of claim 10, wherein said vaccine elicits an immune response against Eastern equine encephalitis virus in the individual, thereby protecting the individual from infections.

13. The method of claim 15, wherein said individual is a human or a domestic animal.

14. A method of protecting an individual from infections resulting from exposure to Eastern equine encephalitis virus, comprising: administering a pharmacologically effective amount of the immunogenic composition of claim 11, wherein the vaccine elicits an immune response against the Eastern equine encephalitis virus in the individual, thereby protecting the individual from the infections.

15. The method of claim 14, wherein said individual is a human or a domestic animal.

16. A method of determining the presence of an antibody to Eastern equine encephalitis virus in a subject, comprising:
obtaining a serum sample from the subject; and
performing an assay using the attenuated virus of claim 8 to determine presence or absence of antigenic reactions, effect on physical properties of the Eastern equine encephalitis virus or a combination thereof in the serum sample, thereby determining the presence of antibody to Eastern equine encephalitis virus in the subject.

17. The method of claim 16, wherein the assays are enzyme linked immunosorbent assays, hemagglutination inhibition assay, complement fixation assay or plaque reduction neutralization assay.

18. The method of claim 16, wherein the subject is a human or a domestic animal.

19. A method of determining the presence of antibody to Eastern equine encephalitis virus in a subject, comprising:
obtaining a serum sample from the subject; and
performing assay using an inactivated Western equine encephalitis virus, wherein the inactivated Western equine encephalitis virus comprises the attenuated virus of claim 8 that is inactivated to determine presence or absence of antigenic reactions, effect on physical properties of the Eastern equine encephalitis virus or a combination thereof in the serum sample, thereby determining the presence of antibody to Eastern equine encephalitis virus in the subject.

20. The method of claim 19, wherein the assays are enzyme linked immunosorbent assays, hemagglutination inhibition assay, complement fixation assay or plaque reduction neutralization assay.

21. The method of claim 19, wherein the subject is a human or a domestic animal.

22. A kit, comprising: the attenuated Eastern equine encephalitis virus of claim 1, the attenuated Eastern equine encephalitis virus of claim 1 that is inactivated or combinations thereof.
